(19) United States
(12) Patent Application Publication
(10) Pub. No.: US 2007/0142472 A1
(43) Pub. Date: Jun. 21, 2007

(54) Process for producing optically active 3-(4-hydroxyphenyl)proprionic acids

(76) Inventors: Tohru Yokozawa, Hiratsuka-shi (JP); Hideo Shimizu, Hiratsuka-shi (JP); Takahiro Fujiwara, Hiratsuka-shi (JP); Yasunori Ino, Hiratsuka-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/578,744

(22) PCT Filed: Nov. 26, 2004
(86) PCT No.: PCT/JP04/17998

§ 371 Date: May 10, 2006

(30) Foreign Application Priority Data
Nov. 27, 2003 (JP) 2003-398201

(51) Int. Cl. A61K 31/192; C07C 65/03

(52) U.S. Cl. 514/570; 562/471

(57) ABSTRACT

The present invention relates to a process for producing an optically active 3-(4-hydroxyphenyl)propionic acid useful as intermediates for medicines, through short steps in good yield and with high optical purity. More specifically, the present invention relates to a process for producing an optically active 3-(4-hydroxyphenyl)propionic acid of the formula (6): wherein R² is an alkyl group; R⁵ to R⁸ are each independently a hydrogen atom or a substituent; and the symbol * is an chiral carbon atom, or a salt thereof, which comprises reacting a benzaldehyde of the formula (1): wherein R¹ is a protective group; and R⁵ to R⁸ are each the same as defined above, with a glycolic acid derivative of the formula (2): wherein R³ is a hydrocarbon group; and R² is the same as defined above, hydrolyzing the resulting product to give a cinnamic acid of the formula (4): wherein R¹, R² and R⁵ to R⁸ are each the same as defined above, or a salt thereof, and subjecting the resulting cinnamic acid (4) or a salt thereof to asymmetric hydrogenation to give an optically active phenylpropionic acid of the formula (5): wherein all the symbols are each the same as defined above, or a salt thereof, followed by deprotection.

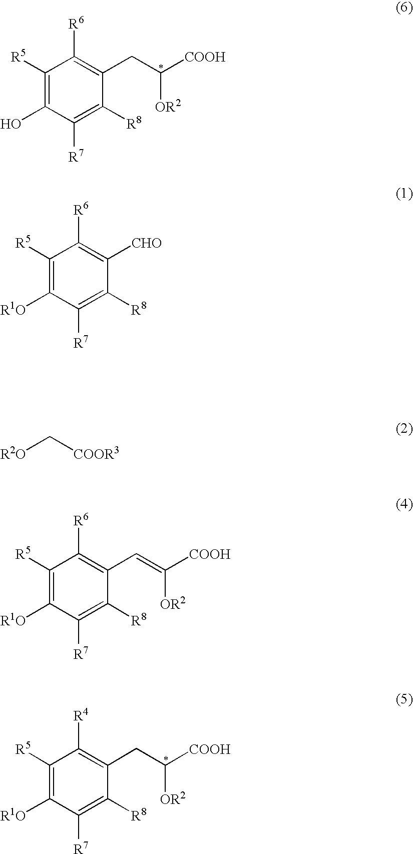

### TECHNICAL FIELD

This invention relates to a process for producing an optically active 3-(4-hydroxyphenyl)propionic acid useful as intermediates for medicines, agrochemicals, etc.

### BACKGROUND ART

Recently, various studies have been made on processes for producing optically active 3-(4-hydroxyphenyl)propionic acids useful as intermediates for medicines, etc.

For example, WO 02/24625 discloses a process for producing (S)-2-alkoxy-3-(4-hydroxyphenyl)propionic acid esters, which comprises reacting L-tyrosine with benzyl chloride to give O-benzyl-L-tyrosine, diazotizing the amino group of the benzylated tyrosine to convert it into the hydroxy group, esterifying and alkylating the carboxy group and the hydroxy group respectively, followed by hydrolysis, converting the resulting(S)-2-alkoxy-3-(4- benzyloxyphenyl)propionic acid with a chiral base into a salt, esterifying the salt, and deprotecting the esterified product.

However, since the method disclosed in WO 02/24625 uses L-tyrosine as a starting material in the reaction, it is required to diazotize the amino group of the starting L-tyrosine. Consequently, such method is not an industrial production method.

There is disclosed a method for preparing (S)-2-ethoxy-3-(4-hydroxyphenyl)propionic acid, which comprises reacting triethyl 2-ethoxyphosphonoacetate with 4-benzyloxy-benzaldehyde, hydrogenating the resulting ethyl 3-(4-benzyloxyphenyl)-2-ethoxyacrylate in the presence of a Pd catalyst and subjecting the resulting racemic ethyl 2-ethoxy-3-(4-hydroxyphenyl)propionate to optical resolution with an enzyme to hydrolyze (S)-form only, in J. Med. Chem. Vol. 46, No. 8, p.1306 (2003) and Organic Process Research & Development, 7(1), p. 82 (2003).

However, the method described in the above literatures has a drawback in that not only hydrolysis and optical resolution using an enzyme have to be carried out after production of racemate, but also selection of enzymes has to be made depending on the substrates, and thus an enzyme capable of hydrolyzing (S)-form selectively has to be used.

U.S. Pat. No. 5,559,267, and J. Am. Chem. Soc., Vol. 120, No. 18, 4345 (1998) disclose a method for asymmetric hydrogenation of α,β-unsaturated carboxylic acid esters wherein the hydroxy group of the a-carbon atom is protected by acetyl or benzoyl, in the presence of a rhodium catalyst and a bisphosphorane ligand.

However, the above method has a problem that metals and ligands to be used are restricted. In addition, since the hydroxy group is protected with acetyl or benzoyl, in order to introduce an alkyl group such as methyl into the hydroxy group, it is necessary and possible to introduce an alkyl group such as methyl only after deprotection of acetyl or benzoyl.

### DISCLOSURE OF THE INVENTION

The present invention has been accomplished in view of the above-mentioned problems, and it is an object of the present invention to provide a process for producing optically active 3-(4-hydroxyphenyl)propionic acids useful as intermediates for medicines, through short steps in high yield and in high optical purity.

As a result of intensive studies on processes for producing optically active 3-(4-hydroxyphenyl)propionic acids by the present inventors, it has been discovered that the objective compounds can be produced through short steps via cinnamic acids as intermediates in high yield and in high optical purity. The present invention has been accomplished on the basis of these findings.

Namely, the present invention is illustrated as following.

1) A process for producing an optically active 3-(4-hydroxyphenyl)propionic acid of the formula (6): 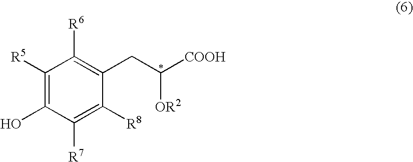
wherein R² is an alkyl group; R⁵ to R⁸ are each independently a hydrogen atom or a substituent; and the symbol * is an chiral carbon atom,
or a salt thereof, which comprises reacting a benzaldehyde of the formula (1): 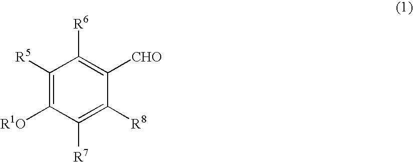
   wherein R¹ is a protective group; and R⁵ to R⁸ are each the same as defined above,
with a glycolic acid derivative of the formula (2): 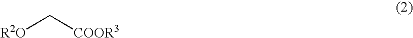
   wherein R³ is a hydrocarbon group; and R² is the same as defined above,
hydrolyzing the resulting product to give a cinnamic acid of the formula (4): 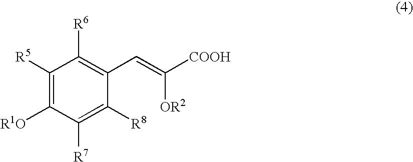
   wherein R¹, R², and R⁵ to R⁸ are each the same as defined above, or a salt thereof, and subjecting the resulting cinnamic acid (4) or a salt thereof to asymmetric hydrogenation to give an optically active phenylpropionic acid of the formula (5): 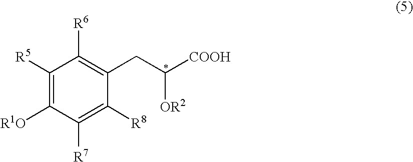
   wherein all the symbols are each the same as defined above, or a salt thereof, followed by deprotection.

2) A process for producing an optically active 3-(4-hydroxyphenyl)propionic acid of the formula (6): 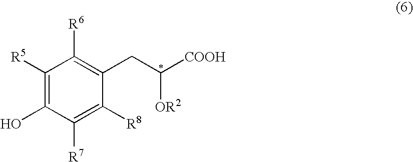
wherein R² is an alkyl group; R⁵ to R⁸ are each independently a hydrogen atom or a substituent; and the symbol * is an chiral carbon atom,
or a salt thereof, which comprises reacting a benzaldehyde of the formula (1): 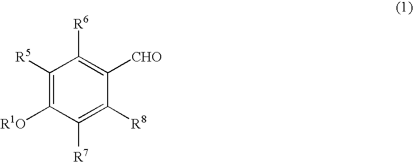
   wherein R¹ is a protective group; and R⁵ to R⁸ are each the same as defined above,
with a glycolic acid derivative of the formula (2): 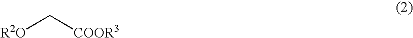
   wherein R³ is a hydrocarbon group; and R²is the same as defined above, followed by hydrolysis to give a cinnamic acid of the formula (4): 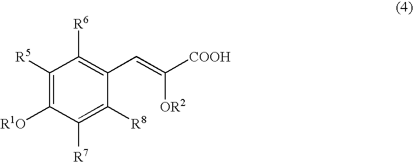
   wherein R¹, R², and R⁵ to R⁸ are each the same as defined above, or a salt thereof, and subjecting the cinnamic acid (4) or a salt thereof to asymmetric hydrogenation. 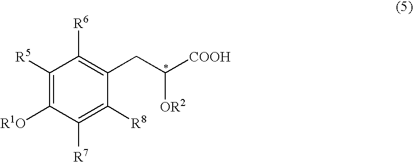
   wherein all the symbols are each the same as defined above, or a salt thereof.

3) A process for producing an optically active 3-(4-hydroxyphenyl)propionic acid of the formula (6): 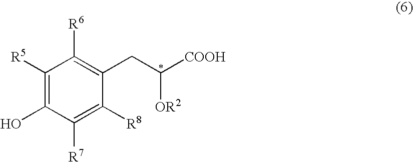
wherein R² is an alkyl group; R⁵ to R⁸ are each independently a hydrogen atom or a substituent; and the symbol * is an chiral carbon atom,
or a salt thereof, which comprises reacting a 4-hydroxybenzaldehyde of the formula (7): 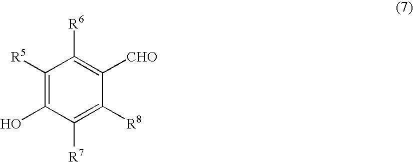
   wherein R⁵ to R⁸ are each the same as defined above, with a glycolic acid derivative of the formula (2): 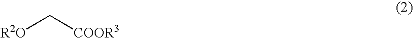
   wherein R³ is a hydrocarbon group; and R² is the same as defined above, followed by hydrolysis to give a 4-hydroxycinnamic acid of the formula (9): 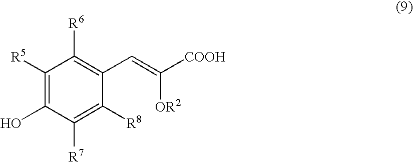
   wherein R², and R⁵ to R⁸ are each the same as defined above, or a salt thereof, and subjecting the 4-hydroxycinnamic acid (9) or a salt thereof to asymmetric hydrogenation.

4) The process according to any one of 1) to 3), wherein the asymmetric hydrogenation is carried out in the presence of a chiral catalyst.

5) The process according to any one of 1) to 4), wherein the chiral catalyst is a transition metal complex.

6) The process according to 4), wherein the transition metal complex is a complex of the metal of Groups 8 to 10 in the periodic table.

7) A process for producing an optically active carboxylic acid of the formula (12): 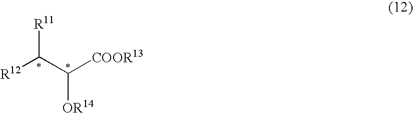
wherein R¹¹ and R¹² are each independently a hydrogen atom or a substituent; R¹³ is a hydrogen atom, an optionally substituted hydrocarbon group or a metal atom; R¹⁴ is a hydrogen atom or a protective group; and the symbol * is an chiral carbon atom, or a salt thereof, which comprises subjecting an α,β-unsaturated carboxylic acid of the formula (11): 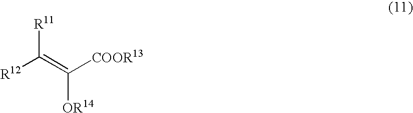
wherein R¹¹ to R¹⁴ are each the same as defined above, or a salt thereof, to asymmetric hydrogenation in the presence of a transition metal complex, provided that when the transition metal complex is rhodium, the protective group represented by R¹⁴ in the above formula (11) is a group other than acyl.

8) The process according to 7), wherein the transition metal complex is a complex of the metal of Groups 8 to 10 in the periodic table.

9) The process according to 1) or 3), wherein the chiral catalyst is a mixture of a chiral ligand and a transition metal compound.

10) The process according to any one of 1) to 3), wherein the optically active phenylpropionic acid of the formula (5) or a salt thereof obtained by the method according to any one of 1) to 3) is crystallized from a solvent.

11) The process according to 10), wherein the solvent used for the crystallization is a member selected from the group consisting of hydrocarbons, alcohols ketones and water, and a mixture thereof.

12) The process according to any one of 1) to 3), wherein the optically active 3-(4-hydroxyphenyl)propionic acid of the formula (6) or a salt thereof obtained by the method according to any one of 1) to 3), is crystallized from a solvent.

13) The process according to 12), wherein the solvent used for the crystallization is a member selected from the group consisting of aromatic hydrocarbons, aliphatic hydrocarbons, alcohols and water, and a mixture thereof.

14) A process for producing an optically active phenylpropionic acid of the formula (5): 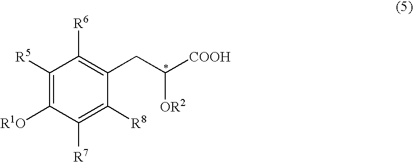
wherein R¹ is a protective group; R² is an alkyl group; R⁵ to R⁸ are each independently a hydrogen atom or a substituent; and the symbol * is an chiral carbon atom,
which comprises subjecting a cinnamic acid of the formula (4): 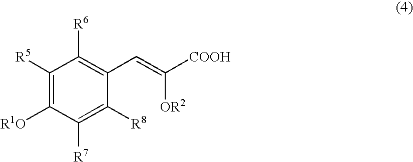
   wherein R¹, R², and R⁵ to R⁸ are each the same as defined above, or a salt thereof,
to asymmetric hydrogenation.

15) A process for producing an optically active 3-(4-hydroxyphenyl)propionic acid of the formula (6): 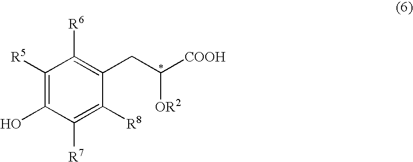
wherein R² is an alkyl group; R⁵ to R⁸ are each independently a hydrogen atom or a substituent; and the symbol * is a chiral carbon atom,
or a salt thereof, which comprises subjecting a cinnamic acid of the formula (4): 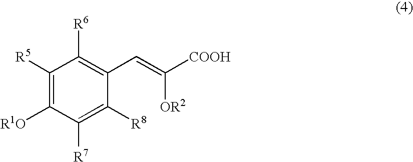
   wherein R¹, R², and R to R⁸ are each the same as defined above, or a salt thereof, to asymmetric hydrogenation.

16) A process for producing an optically active 3-(4-hydroxyphenyl)propionic acid of the formula (6): 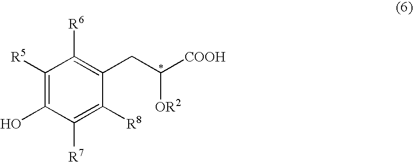
wherein R² is an alkyl group; R⁵ to R⁸ are each independently a hydrogen atom or a substituent; and the symbol * is a chiral carbon atom,
or a salt thereof,
which comprises subjecting a 4-hydroxycinnamic acid of the formula (9): 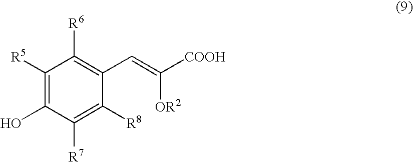
   wherein R², and R⁵ to R⁸ are each the same as defined above, or a salt thereof to asymmetric hydrogenation.

17) A process for producing an optically active 3-(4-hydroxyphenyl)propionic acid of the formula (6): 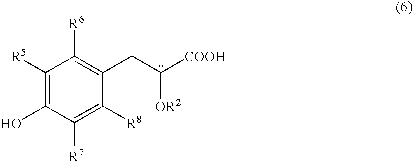
wherein R² is an alkyl group; R⁵ to R⁸ are each independently a hydrogen atom or a substituent; and the symbol * is a chiral carbon atom,
or a salt thereof, and an optically active phenylpropionic acid of the formula (5): 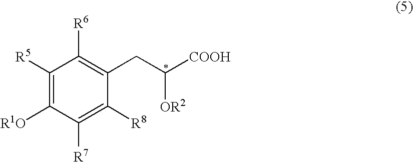
   wherein R¹ is a protective group; and R², R⁵ to R⁸ and the symbols * are each the same as defined above,
or a salt thereof, which comprises subjecting a cinnamic acid of the formula (4): 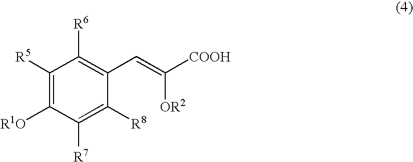
   wherein R¹, R², and R⁵ to R⁸ are each the same as defined above, or a salt thereof, to asymmetric hydrogenation.

18) A process for producing an optically active 3-(4-hydroxyphenyl)propionic acid of the formula (6): 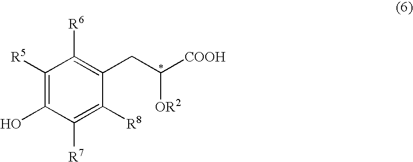
wherein R² is an alkyl group, R⁵ to R⁸ are each independently a hydrogen atom or a substituent; and the symbol * is a chiral carbon atom,
or a salt thereof, which comprises reacting a benzaldehyde of the formula (1): 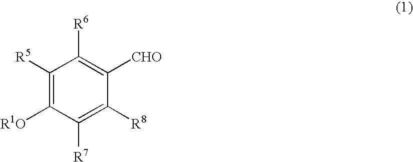
   wherein R¹ is a protective group; and R⁵ to R⁸ are each the same as defined above,
with a glycolic acid derivative of the formula (2): 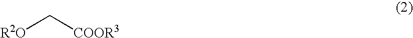
   wherein R³ is a hydrocarbon group, and R² is the same as defined above,
hydrolyzing the resulting product to give a cinnamic acid of the formula (4): 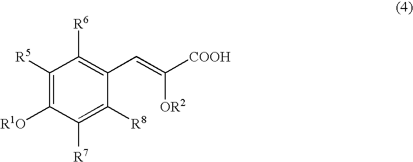
   wherein R¹, R², and R⁵ to R⁸ are each the same as defined above, or a salt thereof, and subjecting the cinnamic acid (4) or a salt thereof to asymmetric hydrogenation to give an optically active phenylpropionic acid of the formula (5): 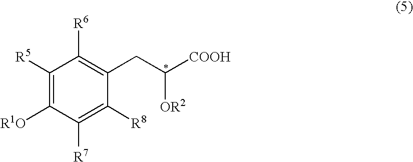
   wherein all the symbols are each the same as defined above, or a salt thereof, and an optically active 3-(4-hydroxyphenyl)propionic acid of the formula (6): 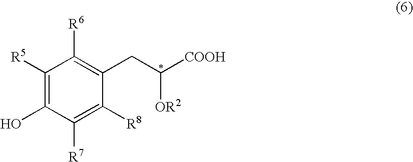
   wherein all the symbols are each the same as defined above, or a salt thereof, followed by deprotection.

The process of the present invention can provide optically active 3-(4-hydroxyphenyl)propionic acids through short steps in high yield and high optical purity.

### THE BEST MODE FOR CARRYING OUT THE INVENTION

As the protective group represented by R¹, there are exemplified those which are-described as hydroxy-protective groups in PROTECTIVE GROUPS IN ORGANIC SYNTHESIS THIRD EDITION (JOHN WILEY & SONS, INC. (1999)). Specific examples of such a hydroxy-protective group include an alkyl group, a substituted alkyl group, an aryl group, a substituted aryl group, an aralkyl group, a substituted aralkyl group, an acyl group, a substituted acyl group, an alkoxycarbonyl group, a substituted alkoxycarbonyl group, an aryloxycarbonyl group, a substituted aryloxycarbonyl group, an aralkyloxycarbonyl group, a substituted aralkyloxycarbonyl group, a heterocyclic group, a substituted heterocyclic group, a substituted silyl group, a sulfonyl group, etc.

The alkyl group may be linear, branched, or cyclic, such as an alkyl group of 1 to 20 carbon atoms, preferably 1 to 10 carbon atoms. Specific examples of such alkyl groups include methyl, ethyl, n-propyl, 2-propyl, n-butyl, 2-butyl, isobutyl, tert-butyl, n-pentyl, 2-pentyl, tert-pentyl, 2-methylbutyl, 3-methylbutyl, 2,2-dimethylpropyl, n-hexyl, 2-hexyl, 3-hexyl, tert-hexyl, 2-methylpenyl, 3-methylpentyl, 4-methylpentyl, 2-methylpentan-3-yl, heptyl, octyl, nonyl, decyl, cyclopropyl, cyclobutyl, cyclopentyl, cyclohexyl, etc.

The aryl group includes, for example, an aryl group with carbon atoms of 6 to 20, and specific examples of such aryl group are phenyl, naphthyl, anthoryl, biphenyl, etc.

The aralkyl group includes, for example, a group wherein at least one hydrogen atom in the aforementioned alkyl group is substituted by the aforementioned aryl group, and such aralkyl group is preferably an aralkyl group of 7 to 20 carbon atoms, including benzyl, 2-phenylethyl, 1-phenylpropyl, 3-naphthylpropyl, etc.

The acyl group may be linear, branched or cyclic. For example, there are mentioned acyl groups of 1 to 20 carbon atoms derived from carboxylic acids such as aliphatic carboxylic acids and aromatic carboxylic acids. Specific examples of such acyl groups include formyl, acetyl, propionyl, butyryl, pivaloyl, pentanoyl, hexanoyl, lauroyl, stearoyl, benzoyl, etc.

The alkoxycarbonyl group may be linear, branched, or cyclic. For example, there are exemplified those of 2 to 20 carbon atoms. Specific examples of such alkoxycarbonyl group include methoxycarbonyl, ethoxycarbonyl, n-propoxycarbonyl, 2-propoxycarbonyl, n-butoxycarbonyl, tert-butoxycarbonyl, pentyloxycarbonyl, hexyloxycarbonyl, 2-ethylhexyloxycarbonyl, lauryloxycarbonyl, stearyloxycarbonyl, cyclohexyloxycarbonyl, etc.

The aryloxycarbonyl group includes, for example, aryloxycarbonyl groups of 7 to 20 carbon atoms, such as phenoxycarbonyl, naphthyloxycarbonyl, etc.

The aralkyloxycarbonyl group includes, for example, aralkyloxycarbonyl groups of 8 to 15 carbon atoms, and specific examples of such aralkyloxycarbonyl groups include benzyloxycarbonyl, phenylethoxycarbonyl, 9-fluorenylmethyloxycarbonyl, etc.

The heterocyclic group includes an aliphatic heterocyclic group and an aromatic heterocyclic group.

The aliphatic heterocyclic group is, for example, a 5- to 8-membered, or more preferably, 5- to 6-membered monocyclic, polycyclic, or fused-ring aliphatic heterocyclic group, which has 2 to 14 carbon atoms and contains as heteroatoms at least one heteroatom, more preferably 1 to 3 heteroatoms, such as nitrogen, oxygen, sulfur atoms, etc. Specific examples of such aliphatic heterocyclic group include, for example, 2-oxo-pyrrolidinyl, piperidino, piperazinyl, morpholino, morpholinyl, tetrahydrofuryl, tetrahydropyranyl, tetrahydrofuranyl, etc.

The aromatic heterocyclic group is, for example, a 5- to 8-membered, more preferably, 5- to 6-membered monocyclic, polycyclic or fused-ring heteroaryl group which is composed of 2 to 15 carbon atoms, and as heteroatoms, at least one heteroatom, and more preferably 1 to 3 heteroatoms such as nitrogen, oxygen, sulfur atoms, etc. Specific examples of such heteroaryl group include, for example, furyl, thienyl, pyridyl, pyrimidyl, pyrazyl, pyridazyl, pyrazolyl, imidazolyl, oxazolyl, thiazolyl, benzofuryl, benzothienyl, quinolyl, isoquinolyl, quinoxalyl, phthalazyl, quinazolyl, naphthyridyl, cinnolyl, benzimidazolyl, benzoxazolyl, benzothiazolyl, acridyl, acridinyl, etc.

The sulfonyl group represented by, for example, the formula R^{a}—SO₂— (R^{a} is a hydrocarbon group, a substituted hydrocarbon group or a substituted amino group). The hydrocarbon group, substituted hydrocarbon group and substituted amino group are each the same as each group which will be defined hereinafter. Specific examples of such sulfonyl group are methanesulfonyl, trifluoromethanesulfonyl, phenylsulfonyl, p-toluenesulfonyl, —SO₂N(CH₃)₂, etc.

The substituted silyl group can be a tri-substituted silyl group, which is formed by substituting three hydrogen atoms of the silyl group by a hydrocarbon group such as alkyl, substituted alkyl, aryl, substituted aryl, aralkyl, substituted aralkyl, alkoxy, substituted alkoxy, substituted silyl, etc. The alkyl, aryl, aralkyl, alkoxy, and substituted silyl groups are each the same as each group hereinbefore mentioned. The substituted alkyl, substituted aryl, substituted aralkyl, and substituted alkoxy groups will be described hereinafter. Specific examples of such substituted silyl group are trimethylsilyl, tert-butyldimethylsilyl, tert-butyldiphenylsilyl, triphenylsilyl, tert-butylmethoxyphenyl, tert-butoxydiphenylsilyl, etc.

The substituted alkyl, substituted aryl, substituted aralkyl, substituted acyl, substituted alkoxycarbonyl, substituted aryloxycarbonyl, substituted aralkyloxycarbonyl, and substituted heterocyclic groups are each the same as those wherein at least one hydrogen atom in each group is substituted by a substituent.

The substituent includes a hydrocarbon group, a substituted hydrocarbon group, a halogen atom, a halogenated hydrocarbon group, a heterocyclic group, a substituted heterocyclic group, an alkoxy group, a substituted alkoxy group, an aralkyloxy group, a substituted aralkyloxy group, an aryloxy group, a substituted aryloxy group, an acyl group, a substituted acyl group, an alkoxy group, a substituted acyloxy group, an alkoxycarbonyl group, a substituted alkoxycarbonyl group, an aryloxycarbonyl group, a substituted aryloxycarbonyl group, an aralkyloxycarbonyl group, a substituted aralkyloxycarbonyl group, an alkylenedioxy group, a nitro group, a substituted amino group, a cyano group, a sulfonyl group, a substituted silyl group, etc.

The hydrocarbon group includes, for example, alkyl, alkenyl, alkynyl, aryl, aralkyl, etc., among which are preferred alkyl, aryl, aralkyl, etc. The alkyl group, aryl group and aralkyl group are each the same as those defined above.

The halogen atom includes fluorine, chlorine, bromine and iodine.

The halogenated hydrocarbon groups are those formed by halogenation such as fluorination, chlorination, bromination, iodination of at least one hydrogen atom of the above-mentioned hydrocarbon groups. Specific examples of such a halogenated hydrocarbon group are alkyl halides such as alkyl halide of 1 to 10 carbon atoms, including. chloromethyl, bromomethyl. 2-chloroethyl, 3-bromopropyl, fluoromethyl, fluoroethyl, fluoropropyl, fluorobutyl, fluoropentyl, fluorohexyl. fluoroheptyl, fluorooctyl, fluorononyl, fluorodecyl, difluoromethyl, difluoroethyl, fluorocyclohexyl, trifluoromethyl, 2,2,2-trifluoroethyl, 3,3,3-trifluoropropyl, pentafluoroethyl, 3,3,4,4,4-pentafluorobutyl, perfluoro-n-propyl, perfluoroisopropyl, perfluoro-n-butyl, perfluoroisobutyl, perfluoro-tert-butyl, perfluoro-sec-butyl, perfluoropentyl, perfluoroisopentyl, perfluoro-tert-pentyl, perfluoro-n-hexyl, perfluoroisohexyl, perfluoroheptyl, perfluorooctyl, perfluorononyl, perfluorodecyl, 2-perfluorooctylethyl, perfluorocyclopropyl, perfluorocyclopentyl, perfluorocyclohexyl, etc.

The alkoxy group may be a linear, branched or cyclic. For example, there is exemplified an alkoxy group of 1 to 20, preferably, 1 to 6 carbon atoms. Specific examples of such alkoxy group include methoxy, ethoxy, n-propoxy, 2-propoxy, n-butoxy, 2-butoxy, isobutoxy, tert-butoxy, n-pentyloxy, 2-methylbutoxy, 3-methylbutoxy, 2,2-dimethylpropyloxy, n-hexyloxy, 2-methylpentyloxy, 3-methylpentyloxy, 4-methylpentyloxy, 5-methylpentyloxy, cyclohexyloxy, etc. As the substituted alkoxy group are mentioned those wherein at least one hydrogen atom in the aforementioned alkoxy group is substituted by a substituent which is described above.

The aryloxy group can be an aryloxy group of 6 to 20 carbon atoms, including, for example, phenyloxy, naphthyloxy, anthryloxy, etc. The substituted aryloxy group can be those wherein at least one hydrogen atom in the above-mentioned aryloxy group is substituted by a substituent which is described above.

The aralkyloxy group can be an aralkyloxy group of 7 to 20 carbon atoms. Specific examples of such aralkyloxy group include benzyloxy, 2-phenylethoxy, 1-phenylpropoxy, 2-phenylpropoxy, 3-phenylpropoxy, 1-phenylbutoxy, 2-phenylbutoxy, 3-phenylbutoxy, 4-phenylbutoxy, 1-phenylpentyloxy, 2-phenylpentyloxy, 3-phenylpentyloxy, 4-phenylpentyloxy, 5-phenylpentyloxy, 1-phenylhexyloxy, 2-phenylhexyloxy, 3-phenylhexyloxy, 4-phenylhexyloxy, 5-phenylhexyloxy, 6-phenylhexyloxy, etc. The substituted aralkyloxy group can be those wherein at least one hydrogen atom in the above-mentioned aralkyloxy group is substituted by a substituent which is described above.

The heterocylic group, acyl group, alkoxycarbonyl group, aryloxycarbonyl group, aralkyloxycarbonyl group, sulfonyl group, and substituted silyl group are each the same as those defined above.

The acyloxy group includes, for example, acyloxy groups of 2 to 20 carbon atoms, derived from carboxylic acids such as aliphatic carboxylic acids, aromatic carboxylic acids, etc. Specific examples of such acyloxy groups are acetoxy, propionyloxy, butyryloxy, pivaloyloxy, pentanoyloxy, hexanoyloxy, lauroyloxy, stearoyloxy, benzoyloxy, etc.

The substituted amino group includes an amino group wherein one or two hydrogen atoms of the amino group is/are substituted by a substituent such as a protective group. Any protective group can be used as far as it can be used as an amino-protective group, and there are exemplified those which are described as an amino-protective group in PROTECTIVE GROUPS IN ORGANIC SYNTHESIS THIRD EDITION (JOHN WILEY & SONS, INC. (1999)). Specific examples of such an amino-protective group are an alkyl group, an aryl group, an aralkyl group, an acyl group, an alkoxycarbonyl group, an aryloxycarbonyl group, an aralkyloxycarbonyl group, a sulfonyl group, etc.

The alkyl, aryl, and aralkyl groups of the above-mentioned amino-protective group are the same with each group of the above-mentioned hydrocarbon groups. Also, the acyl, alkoxycarbonyl, aryloxycarbonyl, and aralkyloxycarbonyl groups are also the same with each group which is mentioned above.

The sulfonyl group as the above-mentioned amino-protective group has the same meaning as those in the above-mentioned substituents.

As the amino groups substituted with an alkyl group, i.e. alkyl-substituted amino groups, there are exemplified mono- and di-alkylamino groups such as N-methylamino, N,N-dimethylamino, N,N-diethylamino, N,N-diisopropylamino, N-cyclohexylamino. etc. The amino group substituted by an aryl group, i.e. aryl-substituted amino group, includes mono- and di-arylamino groups such as N-phenylamino, N,N-diphenylamino, N-naphthylamino, N-naphthyl-N-phenylamino, etc. The amino group substituted with an aralkyl group, i.e. aralkyl-substituted amino group, includes, for example, mono- and di-aralkylamino groups such as N-benzylamino, N,N-dibenzylamino, etc. The amino group substituted by an acyl group, i.e. acylamino group, includes, for example, formylamino, acetylamino, propionylamino, pivaloylamino, pentanoylamino, hexanoylamino, benzoylamino, etc. The amino group substituted with an alkoxycarbonyl group, i.e. alkoxycarbonylamino group, includes, for example, methoxycarbonylamino, ethoxycarbonylamino, n-propoxycarbonylamino, n-butoxycarbonylamino, tert-butoxycarbonylamino, pentyloxycarbonylamino, hexyloxycarbonylamino, etc.

The amino group substituted with an aryloxycarbonyl group, i.e. an aryloxycarbonylamino group, includes, for example, an amino group wherein one hydrogen atom of the amino group is substituted by the above-mentioned aryloxycarbonyl group, and specific examples are phenoxycarbonylamino, naphthyloxycarbonylamino, etc.

The amino group substituted with an aralkyloxycarbonyl group, i.e. an aralkyloxycarbonylamino group includes, for example, benzyloxycarbonylamino, etc.

As the sulfonyl-substituted amino group, there are exemplified —NHSO₂CH₃, —NHSO₂C₆H₅, —NHSO₂C₆H₄CH₃, —NHSO₂CF₃, —NHSO₂N(CH₃)₂, etc.

The alkylenedioxy groups as a substituent are those formed by substituting two adjacent hydrogen atoms in the aromatic ring of the above-mentioned aryl group or aralkyl group, by an alkylenedioxy group. The alkylenedioxy group can be, for example, an alkylenedioxy group of 1 to 3 carbon atoms. Specific examples of such an alkylenedioxy group are methylenedioxy, ethylenedioxy, trimethylenedioxy, propylenedioxy, etc.

The substituted hydrocarbon group, substituted heterocyclic group, substituted alkoxy group, substituted aralkyloxy group, substituted aryloxy group, substituted acyl group, substituted acyloxy group, substituted alkoxycarbonyl group, substituted aryloxycarbonyl group and substituted aralkyloxycarbonyl group can be those wherein at least one hydrogen atom of the above-mentioned hydrocarbon group, heterocyclic group, alkoxy group, aralkyloxy group, aryloxy group, acyl group, acyloxy group, alkoxycarbonyl group, aryloxycarbonyl group, and aralkyloxycarbonyl group is substituted by a substituent mentioned above.

The alkyl group represented by R² may be linear or branched, and includes, for example, an alkyl group of 1 to 4 carbon atoms. Specific examples of such alkyl group are methyl, ethyl, n-propyl, 2-propyl, n-butyl, 2-butyl, isobutyl, tert-butyl, etc.

The hydrocarbon group represented by R³ includes, for example, alkyl, alkenyl, alkynyl, aryl, aralkyl, etc., among which are preferred alkyl, aryl, and aralkyl. The alkyl, aryl, and aralkyl groups are each the same as those mentioned above.

As the substituent represented by R⁵ to R⁸, there are exemplified a hydrocarbon group, a substituted hydrocarbon group, a heterocyclic group, a substituted heterocyclic group. The hydrocarbon group, substituted hydrocarbon group, heterocyclic group, and substituted heterocyclic group have each the same meaning as defined above for R¹ as the protective group.

Specific examples of the benzaldehyde represented by the formula (1) (hereinafter, if required, called as benzaldehyde (1)) include 4-benzyloxybenzaldehyde, 4-tert-butoxybenzaldehyde, 4-benzyloxy-3-methylbenzaldehyde, 4-benzyloxy-3-methoxybenzaldehyde, 4-[2-(9H-acridin-10-yl)ethoxy]benzaldehyde, 4-[3-(4-phenoxyphenoxy)propoxy]benzladehyde, 4-(2-bromoethoxy)benzaldehyde, 4-(2-chloroethoxy)benzaldehyde, 4-(2-chloropropoxy)benzladehyde, 4-(2-iodoethoxy)benzladehyde, 4-(2-iodopropoxy)benzladehyde, 4-(2-hydroxyethoxy)benzaldehyde, 4-(2-hydroxypropoxy)benzaldehyde, etc.

Specific examples of the glycolic acid derivative represented by the formula (2) (hereinafter, if required, called as glycolic acid derivative (2)) include methyl methoxyacetate, ethyl methoxyacetate, propyl methoxyacetate, isopropyl methoxyacetate, butyl methoxyacetate, tert-butyl methoxyacetate, methyl ethoxyacetate, ethyl ethoxyacetate. propyl ethoxyacetate, isopropyl ethoxyacetate, butyl ethoxyacetate, tert-butyl ethoxyacetate, methyl propoxyacetate, ethyl propoxyacetate, propyl propoxyacetate, isopropyl propoxyacetate, butyl propoxyacetate, tert-butyl propoxyacetate, methyl butoxyacetate, ethyl butoxyacetate, propyl butoxyacetate, isopropyl butoxyacetate, butyl butoxyacetate, tert-butyl butoxyacetate, methyl tert-butoxyacetate, ethyl tert-butoxyacetate, propyl tert-butoxyacetate, isopropyl tert-butoxyacetate, butyl tert-butoxyacetate, tert-butyl tert-butoxyacetate, methyl isopropoxyacetate, ethyl isopropoxyacetate, propyl isopropoxyacetate, isopropyl isopropoxyacetate, butyl isopropoxyacetate, tert-butyl isopropoxyacetate, etc.

Specific examples of the cinnamic acid of the formula (4) among the cinnamic acids of the formula (4) or a salt thereof (hereinafter, if required, called as cinnamic acid (4)) in accordance with the present invention include 3-(4-benzyloxyphenyl)-2-methoxyacrylic acid, 3-(4-benzyloxyphenyl).-2-ethoxyacrylic acid, 3-(4-benzyloxyphenyl)-2-propoxyacrylic acid, 3-(4-benzyloxyphenyl)-2-isopropoxyacrylic acid, 3-(4-benzyloxyphenyl)-2-butoxyacrylic acid, 3-(4-benzyloxyphenyl)-2-tert-butoxyacrylic acid, 3-(4-benzyloxyphenyl)-2-tert-butoxyacrylic acid, 3-(4-benzyloxy-3-methoxyphenyl)-2-methoxyacrylic acid, 3-(4-benzyloxy-3-methylphenyl)-2-methoxyacrylic acid, 2-methoxy-3-{4-[3-(4-phenoxyphenoxy)-propoxyphenyllacrylic acid, 3-(4-[2-(9H-acridin-1O-yl)ethoxylphenyl)-2-methoxyacrylic acid, 3-[4-(2-bromoethoxy)phenyl)-2-methoxyacrylic acid, 3-[4-(2-bromopropoxy)phenyl]-2-methoxyacrylic acid, 3-[4-(2-chloroethoxy)phenyl]-2-methoxyacrylic acid, 3-[4-(2-chloropropoxy)phenyl]-2-methoxyacrylic acid, 3-[4-(2-iodoethoxy)phenyl]-2-methoxyacrylic acid, 3-[4-(2-iodopropoxy)phenyl]-2-methoxyacrylic acid, 3-[4-(2-hydroxyethoxy)phenyl]-2-methoxyacrylic acid, 3-[4-(2-hydroxypropoxy)phenyl]-2-methoxyacrylic acid, etc.

As the salt of the cinnamic acid of the formula (4), there re exemplified a metal salt such as alkali metal salts, alkaline earth metal salts, etc. and an ammonium salt. These salts can be a metal salt such as alkali metal salts or alkaline earth metal salts of a cinnamic acid represented by the formula (4-1): 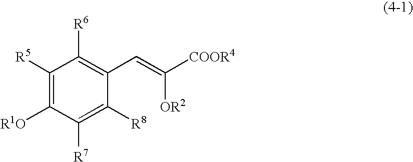
wherein R⁴is a metal atom such as an alkali metal and an alkaline earth metal, and R¹, R², and R⁵ to R⁸ have each the same meaning as defined above, and a cinnamic acid amine salt of the formula (4-2): 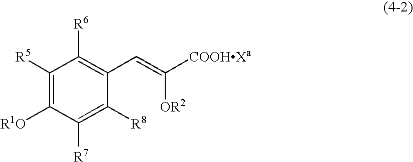
wherein X^{a} is an amine, and R¹R² and R⁵ to R⁸ have each the same meaning as defined above.

The alkali metal represented by R⁴ includes lithium, sodium, potassium, rubidium, caesium, etc.

The alkaline earth metal includes magnesium, calcium, strontium, valium, beryllium, etc.

Examples of the amine represented by X^{a} include ammonia, aliphatic amines such as methylamine, ethylamine, propylamine, butylamine, cyclohexylamine, dimethylamine, diethylamine, diisopropylamine, triethylamine, tripropylamine, diisopropylethylamine, di(2-ethylhexyl)amine, hexadecylamine, tri-n-butylamine, N-methylmorpholine, etc., aromatic amines such as N,N-dimethylaniline, 4-dimethylaminopyridine, etc. and saturated heterocyclic amines such as piperidine, etc.

Specific examples of the metal salts such as alkali metal salts, alkaline earth metal salts, etc. of cinnamic acid of the formula (4-1) include sodium 3-(4-benzyloxyphenyl)-2-methoxyacrylate, lithium 3-(4-benzyloxyphenyl)-2-methoxyacrylate, potassium 3-(4-benzyloxyphenyl)-2-methoxyacrylate, rubidium 3-(4-benzyloxyphenyl)-2-methoxyacrylate, caesium 3-(4-benzyloxyphenyl)-2-methoxyacrylate, beryllium 3-(4-benzyloxyphenyl)-2-methoxyacrylate, magnesium 3-(4-benzyloxyphenyl)-2-methoxyacrylate, potassium 3-(4-benzyloxyphenyl)-2-methoxyacrylate, strontium 3-(4-benzyloxyphenyl)-2-methoxyacrylate, barium 3-(4-benzyloxyphenyl)-2-methoxyacrylate, etc.

Specific examples of the cinnamic acid amine salts of the formula (4-2) include ammonium 3-(4-benzyloxyphenyl)-2-methoxyacrylate, methylammonium 3-(4-benzyloxyphenyl)-2-methoxyacrylate, ethylammonium 3-(4-benzyloxyphenyl)-2-methoxyacrylate, propylammonium 3-(4-benzyloxyphenyl)-2-methoxyacrylate. butylammonium 3-(4-benzyloxyphenyl)-2-methoxyacrylate, cyclohexylammonium 3-(4-benzyloxyphenyl)-2-methoxyacrylate, diethylammonium 3-(4-benzyloxyphenyl)-2-methoxyacrylate, diisopropylammonium 3-(4-benzyloxyphenyl)-2-methoxyacrylate, trimethylammonium 3-(4-benzyloxyphenyl)-2-methoxyacrylate, triethylammonium 3-(4-benzyloxyphenyl)-2-methoxyacrylate, tributylammonium 3-(4-benzyloxyphenyl)-2-methoxyacrylate, pyridinium 3-(4-benzyloxyphenyl)-2-methoxyacrylate, dimethylaminopyridinium 3-(4-benzyloxyphenyl)-2-methoxyacrylate, etc.

Specific examples of the optically active phenylpropionic acid of the formula (5) among the optically active phenylpropionic acid of the formula (5) or a salt thereof (hereinafter, if required, called as optically active phenylpropionic acid (5)) used in the present invention include 3-(4-benzyloxyphenyl)-2-methoxypropionic acid, 3-(4-benzyloxyphenyl)-2-ethoxypropionic acid, 3-(4-benzyloxyphenyl)-2-propoxypropionic acid, 3-(4-benzyloxyphenyl)-2-isopropoxypropionic acid, 3-(4-benzyloxyphenyl)-2-butoxypropionic acid, 3-(4-benzyloxyphenyl)-2-tert-butoxypropionic acid, 3-(4-benzyloxuyphenyl-3-methoxyphenyl)-2-methoxypropionic acid, 3-(4-benzyloxy-3-methylphenyl)-2-methoxypropionic acid, 2-methoxy-3-(4-[3-(4-phenoxyphenoxy)propoxyphenyllpropionic acid, 3-(4-[2-(9H-acridin-10-yl)ethoxy]phenyl)-2-methoxypropionic acid, etc.

As the salt of the optically active phenylpropionic acid of the formula (5), there are exemplified alkali metal salts, alkaline earth metal salts and ammonium salts. These salts can be alkali metal salts or alkaline earth metal salts of an optically active phenylpropionic acid represented, for example, by the formula (5-1): 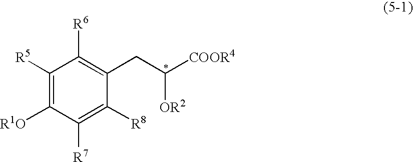
wherein R¹R², R⁴, R⁵ to R⁸ , and * have each the same meaning as defined above, and an optically active phenylpropionic acid amine salt of the formula (5-2): 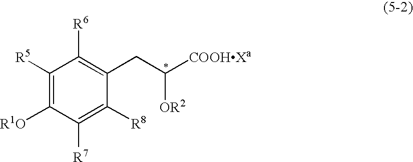
wherein R¹, R², R⁵ to R⁸, X^{a} and * have each the same meaning as defined above.

Specific examples of the metal salts such as alkali metal salts, alkaline earth metal salts, etc. of the optically active phenylpropionic acid of the formula (5-1) include sodium 3-(4-benzyloxyphenyl)-2-methoxypropionate, lithium 3-(4-benzyloxyphenyl)-2-methoxypropionate, potassium 3-(4-benzyloxyphenyl)-2-methoxypropionate, rubidium 3-(4-benzyloxyphenyl)-2-methoxypropionate, caesium 3-(4-benzyloxyphenyl)-2-methoxypropionate, beryllium 3-(4-benzyloxyphenyl)-2-methoxypropionate, magnesium 3-(4-benzyloxyphenyl)-2-methoxypropionate, potassium 3-(4-benzyloxyphenyl)-2-methoxypropionate, strontium 3-(4-benzyloxyphenyl)-2-methoxypropionate, barium 3-(4-benzyloxyphenyl)-2-methoxypropionate, etc.

Specific examples of the amine salt of the optically active phenylpropionic acid of the formula (5-2) include ammonium 3-(4-benzyloxyphenyl)-2-methoxypropionate, methylammonium 3-(4-benzyloxyphenyl)-2-methoxypropionate, ethylammonium 3-(4-benzyloxyphenyl)-2-methoxypropionate, propylammonium 3-(4-benzyloxyphenyl)-2-methoxypropionate, butylammonium 3-(4-benzyloxyphenyl)-2-methoxypropionate, cyclohexylammonium 3-(4-benzyloxyphenyl)-2-methoxypropionate, dimethylammonium 3-(4-benzyloxyphenyl)-2-methoxypropionate, diethylammonium 3-(4-benzyloxyphenyl)-2-methoxypropionate, diisopropylammonium 3-(4-benzyloxyphenyl)-2-methoxypropionate, trimethylammonium 3-(4-benzyloxyphenyl)-2-methoxypropionate, triethylammonium 3-(4-benzyloxyphenyl)-2-methoxypropionate, tributylammonium 3-(4-benzyloxyphenyl)-2-methoxypropionate, pyridinium 3-(4-benzyloxyphenyl)-2-methoxypropionate, dimethylaminopyridinium 3-(4-benzyloxyphenyl)-2-methoxypropionate, etc.

Specific examples of the 4-hydroxybenzaldehyde of the formula (7) (hereinafter, if required, called as 4-hydroxybenzaldehyde (7) in accordance with the present invention include 4-hydroxybenzaldehyde, 2-methyl-4-hydroxybenzaldehyde, 3-methyl-4-hydroxybenzaldehyde, 2-ethyl-4-hydroxybenzaldehyde, 3-ethyl-4-hydroxybenzaldehyde, 2-methoxy-4-hydroxybenzaldehyde, 3-methoxy-4-hydroxybenzaldehyde, 2-nitro-4-hydroxybenzaldenyde, 3-nitro-4-hydroxybenzaldehyde, 25 3-tert-butyl-4-hydroxybenzaldehyde, 2-nitro-4-hydroxybenzaldehyde, 3-tert-butyl-4-hydroxybenzaldehyde, etc.

Specific examples of the 4-hydroxycinnamic acid of the formula (9) among 4-hydroxycinnamic acid of the formula (9) or a salt thereof (hereinafter, if required, called as 4-hydroxycinnamic acid (9)) include 3-(4-hydroxyphenyl)-2-methoxypropionic acid, 3-(4-hydroxyphenyl)-2-ethoxypropionic acid, 3-(4-hydroxyphenyl)-2-propoxypropionic acid, 3-(4-hydroxyphenyl)-2-isopropoxypropionic acid, 3-(4-hydroxyphenyl)-2-butoxypropionic acid, 3-(4-hydroxyphenyl)-2-tert-butoxypropionic acid, etc.

As the salt of the 4-hydroxycinnamic acid of the formula (9), there are exemplified metal salts such as alkali metal salts, alkaline earth metal salts, etc. and ammonium salts. These salts can be a metal salt such as alkali metal salts and alkaline earth metal salts of the 4-hydroxycinnamic acid represented by the formula (9-1): 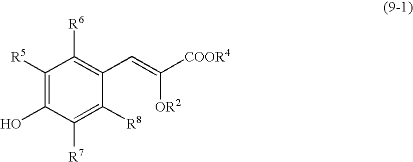
wherein R², R⁴, and R⁵ to R⁸ have each the same meaning as defined above, and a 4-hydroxycinnamic acid amine salt of the of the formula (9-2);: 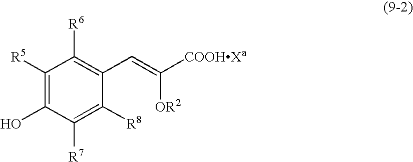
wherein R², R⁵, to R⁸and X^{a} have each the same meaning as defined 20 above.

Specific examples of the metal salts such as alkali metal salts and alkaline earth metal salts of the 4-hydroxycinnamic acid of the formula (9-1) include sodium 3-(4-hydroxyphenyl)-2-methoxyacrylate, lithium 3-(4-hydroxyphenyl)-2-methoxyacrylate, potassium 3-(4-hydroxyphenyl)-2-methoxyacrylate, rubidium 3-(4-hydroxyphenyl)-2-methoxyacrylate, caesium 3-(4-hydroxyphenyl)-2-methoxyacrylate, beryllium 3-(4-hydroxyphenyl)-2-methoxyacrylate, magnesium 3-(4-hydroxyphenyl)-2-methoxyacrylate, calcium 3-(4-hydroxyphenyl)-2-methoxyacrylate, strontium 3-(4-hydroxyphenyl)-2-methoxyacrylate, barium 3-(4-hydroxyphenyl)-2-methoxyacrylate, etc.

Specific examples of the 4-hydroxycinnamic acid amine salts of the formula (9-2) include ammonium 3-(4-hydroxyphenyl)-2-methoxyacrylate, methylammonium 3-(4-hydroxyphenyl)-2-methoxyacrylate, ethylammonium 3-(4-hydroxyphenyl)-2-methoxyacrylate, propylammonium 3-(4-hydroxyphenyl)-2-methoxyacrylate, lbutylammonium 3-(4-hydroxyphenyl)-2-methoxyacrylate, cyclohexylammonium 3-(4-hydroxyphenyl)-2-methoxyacrylate, dimethylammonium 3-(4-hydroxyphenyl)-2-methoxyacrylate, diethylammonium 3-(4-hydroxyphenyl)-2-methoxyacrylate, diisopropylammonium 3-(4-hydroxyphenyl)-2-methoxyacrylate, trimethylammonium 3-(4-hydroxyphenyl)-2-methoxyacrylate, triethylammonium 3-(4-hydroxyphenyl)-2-methoxyacrylate, tributylammonium 3-(4-hydroxyphenyl)-2-methoxyacrylate, pyridinium 3-(4-hydroxyphenyl)-2-methoxyacrylate, dimethylaminopyridinium 3-(4-hydroxyphenyl)-2-methoxyacrylate, etc.

Specific examples of the optically active 3-(4-hydroxyphenyl)propionic acid of the formula (6) among the the optically active 3-(4-hydroxyphenyl)propionic acid of the formula (6) or a salt thereof (hereinafter, if required, called as 3-(4-hydroxyphenyl)propionic acid (6)) obtained in the present invention include 3-(4-hydroxyphenyl)-2-methoxypropionic acid, 3-(4-hydroxyphenyl)-2-ethoxypropionic acid, 3-(4-hydroxyphenyl)-2-propoxypropionic acid, 3-(4-hydroxyphenyl)-2-isopropoxypropionic acid, 3-(4-hydroxyphenyl)-2-butoxypropionic acid, 3-(4-hydroxyphenyl)-2-tert-butoxypropionic acid, etc.

As the salt of the optically active 3-(4-hydroxyphenyl)propionic acid of the formula (6), there are exemplified metal salts such as alkali metal salts, alkaline earth metal salts, etc. and ammonium salts. These salts can be metal salts such as alkali metal salts or alkaline earth metal salts of the optically active 3-(4-hydroxyphenyl)propionic acid of the formula (6-1): 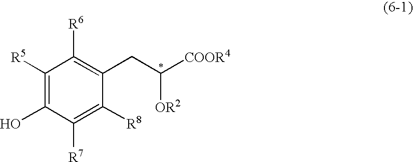
wherein R², R⁴, R⁵ to R⁸ and * have each the same meaning as defined above, and an optically active 3-(4-hydroxyphenyl)propionic acid amine salt of the formula (6-2): 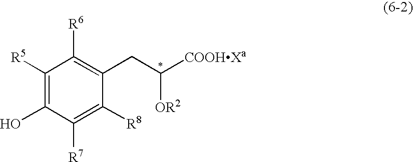
wherein R², R⁵ to R⁸, X^{a} and * have each the same meaning as defined above.

Specific examples of the metal salts such as alkali metal salts, alkaline earth metal salts, etc. of the optically active 3-(4-hydroxyphenyl) propionic acid of the formula (6-1) include sodium 3-(4-hydroxyphenyl)-2-methoxypropionate, lithium 3-(4-hydroxyphenyl)-2-methoxypropionate, potassium 3-(4-hydroxyphenyl)-2-methoxypropionate, rubidium 3-(4-hydroxyphenyl)-2-methoxypropionate, caesium 3-(4-hydroxyphenyl)-2-methoxypropionate, beryllium 3-(4-hydroxyphenyl)-2-methoxypropionate, magnesium 3-(4-hydroxyphenyl)-2-methoxypropionate, calcium 3-(4-hydroxyphenyl)-2-methoxypropionate, strontium 3-(4-hydroxyphenyl)-2-methoxypropionate, barium 3-(4-hydroxyphenyl)-2-methoxypropionate, etc.

Specific examples of the optically active 3-(4-hydroxyphenyl)propionic acid amine salts of the formula (6-2) include ammonium 3-(4-hydroxyphenyl)-2-methoxypropionate, methylammonium 3-(4-hydroxyphenyl)-2-methoxypropionate, ethylammonium 3-(4-hydroxyphenyl)-2-methoxypropionate, propylammonium 3-(4-hydroxyphenyl)-2-methoxypropionate, butylammonium 3-(4-hydroxyphenyl)-2-methoxypropionate, cyclohexylammonium 3-(4-hydroxyphenyl)-2-methoxypropionate, dimethylammonium 3-(4-hydroxyphenyl)-2-methoxypropionate, diethylammonium 3-(4-hydroxyphenyl)-2-methoxypropionate, diisopropylammonium 3-(4-hydroxyphenyl)-2-methoxypropionate, trimethylammonium 3-(4-hydroxyphenyl)-2-methoxypropionate, triethylammonium 3-(4-hydroxyphenyl)-2-methoxypropionate, tributylammonium 3-(4-hydroxyphenyl)-2-methoxypropionate, pyridinium 3-(4-hydroxyphenyl)-2-methoxypropionate, dimethylaminopyridinium 3-(4-hydroxyphenyl)-2-methoxypropionate, etc.

The production process of the present invention will be illustrated by the following reaction scheme.

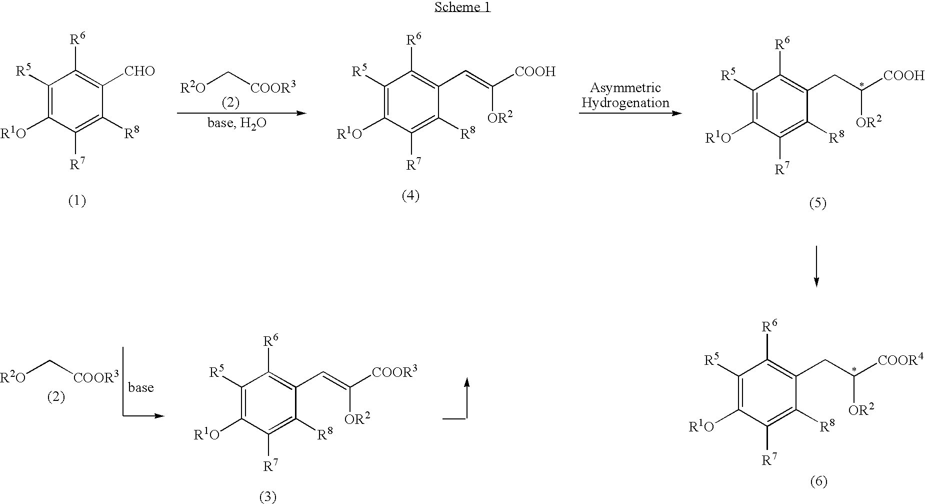

The cinnamic acid of the formula (4) or a salt thereof can be produced by reacting a benzaldehyde of the formula (1) with a glycolic acid derivative (2) in a suitable solvent in the presence of a base, followed by hydrolysis.

The amount of the glycolic acid derivative (2) to be used is usually selected appropriately from the range of 1 to 10 equivalents, preferably 1 to 5 equivalents to the benzaldehyde of the formula (1).

Examples of the solvent include, for example, aliphatic hydrocarbons such as pentane, hexane, heptane, octane, decane, cyclohexane, etc.; aromatic hydrocarbons such as benzene, toluene, xylene, etc.; halogenated hydrocarbons such as dichloromethane, 1,2-dichloroethane, chloroform, carbon tetrachloride, o-dichlorobenzene, etc.; ethers such as diethyl ether, diusopropyl ether, tert-butyl methyl ether, dimethoxyethane, ethyleneglycol diethyl ether, tetrahydrofuran, 1,4-dioxane, 1,3-dioxolane, etc.; ketones such as acetone, methyl ethyl ketone, methyl isobutyl ketone, cyclohexanone, etc.; alcohols such as methanol, ethanol, 2-propanol, n-butanol, 2-ethoxyethanol, etc.; polyalcohols such as ethylene glycol, propylene glycol, 1,2-propanediol, glycerol, etc.; sulfoxides such as dimethyl sulfoxide, etc.; amides such as N,N-dimethylformamide, formamide, N,N-dimethylacetamide, etc.; cyano-containing organic compounds such as acetonitrile, etc., etc. These solvents may be used alone or appropriately in combination of two or more kinds of them.

The amount of the solvent used is usually selected appropriately from the range of 0.1-fold to 100-fold amount, preferably 1-fold to 20-fold amount to the benzaldehyde (1).

As the base are exemplified inorganic bases and organic bases. The inorganic base includes potassium carbonate, potassium hydroxide, lithium hydroxide, sodium bicarbonate, sodium carbonate, potassium bicarbonate, sodium hydroxide, magnesium carbonate, calcium carbonate, etc. Theorganic base includes alkali metal/alkaline earth metal salts such as potassium methoxide, sodium methoxide, lithium methoxide, sodium ethoxide, potassium isopropoxide, potassium tert-butoxide, potassium naphthalenide, sodium acetate, potassium acetate, mangensium acetate, calcium acetate, etc.; organic amines such as triethylamine, diisopropylethylamine, N,N-dimethylaniline, piperidine, pyridine, 4-dimethylaminopyridine, 1,5-diazabicyclo[4.3.0]non-5-ene, 1,8-diazabicyclo[5.4.0]undec-7-ene, tri-n-butylamine, N-methylmorpholine, etc.; metal hydride complexes such as sodium hydride, sodium borohydride, aluminum lithium hydride, etc.; organometal compounds such as methyl magnesium bromide, ethyl magnesium bromide, propyl magnesium bromide, methyllithium, ethyllithium, propyllithium, n-butyllithium, tert-butyllithium, etc. and quaternary ammonium salts.

The amount of the base used is usually selected appropriately from the range of 0.01 to 10 equivalents, preferably 1 to 5 equivalents, to the glycolic acid derivative (2).

The reaction temperature is usually selected appropriately from the range of 0° C. to the boiling point of the solvent used, preferably 20° C. to 80° C.

The reaction time is usually selected appropriately from the range of 0.1 to 48 hours, preferably 1 to 10 hours.

The reaction between the benzaldehyde (1) and the glycolic acid derivative (2) may be carried out by isolating, after optional post-treatment and purification or subjecting to the subsequent reaction without post-treatment and purification, a cinnamic acid ester of the formula (3): 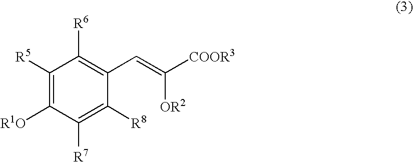
wherein R¹, R², R^{3,} and R⁵ to R⁸ have each the same meaning as defined above (hereinafter, if required, called as cinnamic acid ester (3)), followed by hydrolysis, thereby giving a cinnamic acid (4). Alternatively, without isolation of the cinnamic acid ester (3), hydrolysis may be carried out upon addition of water, alcohol and/or the above-mentioned base.

The hydrolysis may be carried out by a method usually employed in the art.

The hydrolysis, for example, may be conducted by treating the cinnamic acid ester (3) in an alcohol in the presence of an aqueous alkaline solution of the above-mentioned base such as lithium hydroxide, sodium hydroxide, potassium hydroxide, etc., or in a mixture of the alcohol and the above-mentioned base such as lithium hydroxide, sodium hydroxide, potassium hydroxide, etc.

Examples of the alcohol include, for example, methanol, ethanol, 2-propanol, n-butanol, 2-ethoxyethanol and the like.

The amount of the base is usually selected appropriately from the range of 0.1 to 10-fold amount, preferably 1 to 5-fold amount to the cinnamic acid ester (3).

The amount of water is usually selected appropriately from the range of 0.1 to 100-fold amount, preferably 1 to 20-fold amount to the cinnamic acid ester (3).

The amount of alcohol is usually selected appropriately from the range of 0.1 to 100-fold amount, preferably 1 to 20-fold amount to the cinnamic acid ester (3).

The hydrolysis temperature is usually selected appropriately from the range of 0° C. to boiling point of the solvent, preferably 20 to 60° C.

The hydrolysis time is usually selected appropriately from the range of 0.5 to 10 hours, preferably 1 to 5 hours.

Specific examples of the cinnamic acid ester (3) include methyl 3-(4-benzyloxyphenyl)-2-methoxyacrylate, ethyl 3-(4-benzyloxyphenyl)-2-methoxyacrylate, propyl 3-(4-benzyloxyphenyl)-2-methoxyacrylate, butyl 3-(4-benzyloxyphenyl)-2-methoxyacrylate, tert-butyl 3-(4-benzyloxyphenyl)-2-methoxyacrylate, methyl 3-(4-benzyloxyphenyl)-2-ethoxyacrylate, ethyl 3-(4-benzyloxyphenyl)-2-ethoxyacrylate, propyl 3-(4-benzyloxyphenyl)-2-ethoxyacrylate, butyl 3-(4-benzyloxyphenyl)-2-ethoxyacrylate, tert-butyl 3-(4-benzyloxyphenyl)-2-ethoxyacrylate, methyl 3-(4-benzyloxyphenyl)-2-propoxyacrylate, ethyl 3-(4-benzyloxyphenyl)-2-propoxyacrylate, propyl 3-(4-benzyloxyphenyl)-2-propoxyacrylate, butyl 3-(4-benzyloxyphenyl)-2-propoxyacrylate, tert-butyl 3-(4-benzyloxyphenyl)-2-propoxyacrylate, methyl 3-(4-benzyloxyphenyl)-2-butoxyacrylate, ethyl 3-(4-benzyloxyphenyl)-2-butoxyacrylate, propyl 3-(4-benzyloxyphenyl)-2-butoxyacrylate, butyl 3-(4-benzyloxyphenyl)-2-butoxyacrylate, tert-butyl 3-(4-benzyloxyphenyl)-2-butoxyacrylate, etc.

The resulting cinnamic acid of the formula (4) or its salt may be a mixture of a cinnamic acid of free carboxy group of the formula (4) and a metal salt of a cinnamic acid of the formula (4-1) and/or a cinnamic acid amine salt of the formula (4-2).

Further, the obtained cinnamic acid of the formula (4) is, if required, converted into a metal salt of a cinnamic acid of the formula (4-1) or an amine salt of a cinnamic acid of the formula (4-2), or a salt different from a salt of the cinnamic acid of the formula (4), using an aqueous solution of the above-mentioned base.

Moreover, the cinnamic acid (4) may be subjected to post-treatment, if required, or to the subsequent reaction without any post-treatment and isolation.

The optically active phenylpropionic acid (5) can be produced by asymmetric hydrogenation of the cinnamic acid (4).

The asymmetric hydrogenation may be carried out in the presence of a chiral catalyst to give an optically active phenylpropionic acid (5) in good yield with high optical purity. The chiral catalyst is preferably a catalyst for asymmetric hydrogenation.

As the catalyst for asymmetric hydrogenation, it is preferred to use a chiral transition metal complex. The chiral transition metal complex can be preferably a complex containing a transition metal and a chiral ligand. Said transition metal complex may be used in situ for hydrogenation.

The transition metal in the above-mentioned transition metal complex is preferably a metal of Groups 8 to 10 in the periodic table.

As the transition metal complex, there are exemplified compounds represented by the formula (13) or (14):
MₘLₙXₚY_{q} (13)
[MₘLₙXₚY_{q}]Zₛ (14)

In the above formulae (13) and (14), wherein M is a transition metal of Groups 8 to 10 in the periodic table; L is a chiral ligand; X is a halogen atom, a carboxylate group, an allyl group, a 1,5-cyclooctadiene group or a norbornadiene group; Y is a ligand; Z is an anion or a cation; and m, n, p, q and s are each an integer of 0 to 5.

The transition metals of Groups 8 to 10 of the periodic table represented by M in the formulae (13) and (14) are each the same or different, and include ruthenium (Ru), rhodium (Rh), iridium (Ir), palladium (Pd), nickel (Ni), etc.

The chiral ligand represented by L may be the same or different monodentate or bidentate ligand. Preferable chiral ligand can be an optically active phosphine ligand, and more preferable chiral ligand can be an optically active bidentate phosphine ligand.

The optically active bidentate ligand can be, for example, phosphine compounds represented by the formula (15):
R²¹R²²P-Q-PR²³R²⁴ (15)
wherein R²¹ to R²⁴ are each independently a hydrocarbon group, a substituted hydrocarbon group, a heterocyclic group or a substituted heterocyclic group; and Q is a spacer.

As the hydrocarbon group, substituted hydrocarbon group, heterocyclic group or substituted heterocyclic group represented by R²¹ to R²⁴, they may have the same meaning as defined above for each group of R¹, R⁵ to R⁸ in the formula (1).

As the spacer represented by Q there are exemplified optionally substituted divalent organic groups such as alkylene groups and arylene groups.

The alkylene group includes, for example an alkylene group of 1 to 6 carbon atoms, and specific examples of such group include methylene, ethylene, trimethylene, propylene, tetramethylene, pentamethylene, hexamethylene, etc. The arylene group includes, for example, an arylene group of 6 to 20 carbon atoms, and specific examples of such arylene group are phenylene, biphenyldiyl, binaphthalenediyl, etc. These divalent organic groups may be substituted by the above-mentioned substituent.

The above-mentioned divalent organic group may contain at least one oxygen atom, carbonyl group, etc., at an arbitrary position of the terminal or the chain, in the aforementioned groups.

Specific examples of such chiral ligand include cyclohexylanisylmethylphosphine (CAMP), 1,2-bis(anisylphenylphosphino)ethane( DIPAMP), 1,2-bis(alkylmethylphosphino)ethane (BisP*), 2,3-bis(diphenylphosphino)butane (CHIRAPHOS), 1,2-bis(diphenylphosphino)propane( PROPHOS), 2,3-bis(diphenylphosphino)-5-norbornene (NORPHOS), 2,3-O-isopropylidene-2,3-dihydroxy-1,4-bis(diphenylphosphino)butane (DIOP), 1-cyclohexyl-1,2-bis(diphenylphosphino)ethane (CYCPHOS), 1-substituted-3,4-bis(diphenylphosphino)pyrrolidine (DEGPHOS), 2,4-bis(diphenylphosphino)pentane(SKEWPHOS), 1,2-bis(substituted phospholano)benzene (DuPHOS), 1,2-bis(substituted phospholano)ethane (BPE), 1-(substituted phospholano)-2-(diphenylphosphino)benzene (UCAP-Ph), 1-(bis(3, 5-dimethylphenyl)phosphino)-2-(substituted phospholano)benzene (UCAP-DM), 1-(substituted phospholano)-2-(bis(3,5-di(t-butyl)-4-methoxyphenyl)phosphino)benzene (UCAP-DTBM), 1-(substituted phospholano)-2-(di-naphthalen-1- yl-phosphino)benzene (UCAP-(1-Nap)), 1-[1′,2-bis(diphenylphosphino)ferrocenyl]ethylamine (BPPFA), 1-[1′,2-bis(diphenylphosphino)ferrocenyllethyl alcohol (BPPFOH), 2,2′-bis(diphenylphosphino)-1,1′-dicyclopentane(BICP), 2,2′-bis(diphenylphosphino)-1,1′-binaphthyl (BINAP), 2,2′-bis(diphenylphosphino)-1,1′-(5,5′,6,6′,7,7′,8,8′-octa-hydrobinaphthyl) (H₈-BINAP), 2,2′-bis(di-p-tolylphosphino)-1,1′-binaphthyl(TOL-BINAP), 2,2′-bis(di(3,5-dimethylphenyl)phosphino)-1,1′-binaphthyl (DM-BINAP), 12,2′-bis(diphenylphosphino)-6,6′-dimethyl-1,1′-biphenyl (BICHEP), [4,4′-bi-1,3-benzodioxole]-5,5′-diylbis[diphenylphosphine] (SEGPHOS), [4,4′-bi-1,3-benzodioxole]-5,5′-diylbis[bis(3,5-dimethylphenyl)phosphine](DM-SEGPHOS), [(4S)-[4,4′-bi-1,3-benzodioxolel-5,5′-diyl]bis(bis[3,5-bis (1,1-dimethylethyl)-4-methoxyphenyl]phosphine](DTBM-SEGPHOS), etc.

A bis-heterocyclic compound may be used as the chiral ligand other than the above-mentioned optically active bidentate ligand.

The ligands represented by Y are each, the same or different, neutral ligands such as aromatic compounds and olefinic compounds, amines and so on. Examples of the aromatic compound include benzene, p-cymene, 1,3,5-trimethylbenzene (mesitylene), hexamethylbenzene, etc.; examples of the olefinic compound include ethylene, 1,5-cyclooctadiene, cyclopentadiene, norbornadiene, etc.; and examples of the other neutral ligand include N,N-dimethylformamide (DMF), acetonitrile, benzonitrile, acetone, chloroform, etc.

Examples of the amines include diamines such as 1,2-diphenylethylenediamine (DPEN), 1,2-cyclohexylethylenediamine, 1,2-diaminocyclohexane, ethylenediamine, 1,1-bis(4-methoxyphenyl)-2-isopropylethylenediamine (DAIPEN), and the like, an aliphatic amines such as triethylamine and the like, and an aromatic amines such as pyridine and the like.

Halogen atom represented by X includes chlorine atom, bromine atom and iodine atom.

In the formula (14), Z represents an anion or a cation. Examples of Z anion include BF₄, ClO₄, OTf, PF₆, SbF₆, BPh₄, Cl, Br, I, I₃, sulfonate, etc., wherein Tf means triflate group (SO₂CF₃).

The cation can be represented, for example by the following formula: [(R)₂NH₂]⁺ wherein a couple of R are each, the same or different, a hydrogen atom or an optionally substituted hydrocarbon group.

In the above formula, the optionally substituted hydrocarbon groups represented by R is the same as the aforementioned optionally substituted hydrocarbon group. The optionally substituted hydrocarbon group represented f by R can be preferably an alkyl group of 1 to 5 carbon atoms, a cycloalkyl group, an optionally substituted phenyl group or an optionally substituted benzyl group.

Specific examples of the cation of the above formula include, for example, [Me₂NH₂]⁺, [Et₂NH₂]⁺, [Pr₂NH²]⁺, etc.

The following is the detailed explanation about preferable embodiments of the aforementioned transition metal complexes.

[1] Formula (13)
MₘLₙXₚY_{q} Formula (13)
1) When M is Ir or Rh, X is Cl, Br or I, and when L is amonodentate ligand, then m=p=2, n=4 and q=0; and when L is a bidentate ligand, then m=n=p=2 and q=0.
2) When M is Ru, (i) X is Cl, Br, or I, and Y is a trialkylamino group, and when L is a monodentate ligand, then m=2, n=p=4 and q=1; and when L is a bidentate ligand, then m=n=2, p=4 and q=1.

(ii) X is Cl, Br or I, and Y is a pyridyl group or a ring-substituted pyridyl group, and when L is a monodentate ligand, then m=1, n=p=2 and q=2; and when L is a bidentate ligand, then m=n=1, p=2 and q=2,

(iii) X is a carboxylato group, and when L is a monodentate ligand, then m=1, n=p=2, and q=0; and when L is a bidentate ligand, then m=n=1, p=2, and q=0, and

(iv) X is Cl, Br or I, and when L is a monodentate ligand, then m=p=2, n=4 and q=0; and when L is a bidentate ligand, then m=n=p=2 and q=0.
3) When M is Pd, (i) X is Cl, Br or I, and when L is a monodentate ligand, then m=1, n=2, p=2 and q=0; and when L is a bidentate ligand, then m=n=1, p=2 and q=0 and

(ii) X is an allyl group, and when L is a monodentate ligand, then m=p=2, n=4 and q=0; and when L is a bidentate ligand, then m=n=p=2 and q=0.
4) When M is Ni, X is Cl, Br or I, and when L is a monodentate ligand, then m=1, n=2, p=2 and q=0; and when L is a bidentate ligand, then m=n=1, p=2 and q=0.
   [2] Formula (14)
   [MₘLₙXₚY_{q}]Zₛ (14)
1) When M is Ir or Rh, then X is 1,5-cyclooctadiene or norbornadiene, Z is BF₄, ClO₄, OTf, PF₆, SbF₆ or BPh₄, m=n=p=s=1 and q=0, or m=s=1, n=2 and p=q=0.
2) When M is Ru, (i) X is Cl, Br or I, Y is a neutral ligand such as an aromatic compound and an olefinic compound, and Z is Cl, Br, I, I₃orsulfonate, and when L is amonodentate ligand, then m=p=s=q=1 and n=2; and when L is a bidentate ligand, then m=n=p=s=q=1.

(ii) Z is BF₄, ClO₄, OTf, PF₆, SbF₆ or BPh₄, and when L is a monodentate ligand, then m=1, n=2, p=q=0 and s=2; and when L is a bidentate ligand, then m=n=1, p=q=0 and s=2 and

(iii) When Z is an ammonium ion and L is a bidentate ligand, then m=2, n=2, p=5 and q=0.
3) When M is Pd or Ni, (i) Z is BF₄, ClO₄, OTf, PF₆, SbF₆ or BPh₄ and when L is a monodentate ligand, then m=1, n=2, p=q=0, s=2; and when L is a bidentate ligand, then m=n=1, p=q=0 and s=2.

These transition metal complexes can be produced by using conventional methods.

In the formulae of the transition metal complexes given below, the meanings of the symbols used are as follows, L: a chiral ligand; cod: 1,5-cyclooctadiene; nbd: norbornadiene; Tf: triflate group (SO₂CF₃); Ph: phenyl group; and Ac: acetyl group. As specific examples of such transition metal complexes, only the transition metal complexes in which bidentate ligands are used as the chiral ligand are shown in order to avoid complication.

Rhodium Complex:

The rhodium complex can be produced according to the method described in “JIKKEN KAGAKU KOZA, 4^{th} Ed., Volume 18, Organic Metal Complexes, pp. 339-344, published by Maruzen, in 1991”. More specifically, rhodium complex can be produced by reacting bis(cycloocta-1,5-diene)rhodium(I) tetrafluoroborate with a chiral ligand.

Specific examples of the rhodium complex include, for example, those given below: [Rh(L)Cl]₂, [Rh(L)Br]₂, [Rh(L)I]₂, [Rh(cod)(L)]BF₄, [Rh(cod)(L)]ClO₄, [Rh(cod)(L)]PF₆, [Rh(cod)(L)]BPh₄, [Rh(cod)(L)]OTf, [Rh(nbd)(L)]BF₄, [Rh(nbd)(L)]ClO₄, [Rh(nbd)(L)]PF₆, [Rh(nbd)(L)]BPh₄ [Rh(nbd)(L)]OTf, [Rh(L)₂]ClO₄, [Rh(L)₂]PF₆, [Rh(L)₂]OTf and [Rh(L)₂]BF₄.

Ruthenium Complex:

The ruthenium complex can be obtained according to the method described in the literature (T. Ikariya et al., J. Chem. Soc., Chem. Commun., 1985, 922) and in other literatures. More specifically, the ruthenium complex can be produced by heating [Ru(cod)Cl₂]n and a chiral ligand under reflux in toluene as solvent in the presence of triethylamine.

The ruthenium complex can also be produced according to the method described in the literature (K. Mashima et al., J. Chem. Soc., Chem. Commun., 1989, 1208). More specifically, the ruthenium complex can be obtained by heating [Ru(p-cymene)I₂]₂ and a chiral ligand in methylene chloride and ethanol with stirring. Specific examples of such ruthenium complex include, for example, those given below: Ru(OAc)₂(L), Ru₂Cl₄(L)₂NEt₃, (RuCl(benzene)(L)]Cl, [RuBr(benzene)(L)]Br, [RuI(benzene)(L)]I, [RuCl(p-cymene)(L)]Cl, [RuBr(p-cymene)(L)]Br, [RuI(p-cymene)(L)]I, [Ru(L)](BF₄)₂, [Ru(L)](ClO₄)₂, [Ru(L)](PF₆)₂, [Ru(L)](BPh₄)₂, [Ru(L)](OTF)₂, Ru(OCOCF₃)₂(L), [{RuCl(L)₂}(μ-Cl)₃][Me₂NH₂], [{RuCl(L)}₂(μCl)₃][Et₂NH₂] Ru(OCOCF₃)₂(L), [{RuCl(L)₂}(μ-Cl)₃][Me₂NH₂], {RuBr(L)₂}(μ-Cl)₃][Me₂NH₂], {RuBr(L)₂}(μ-Cl)₃][Et₂NH₂], RuCl₂(L), RuBr₂(L), RuI₂(L), RuCl₂(L)(diamine), RuBr₂(L)(diamiine), RuI₂(L) (diamine), [{RuI(L)}₂(μ-I)₃][Me₂NH₂], [{RuI(L)}₂(μ-I)₃][Et₂NH₂], RuCl₂(L) (pyridine), RuBr₂(L) (pyridine) and RuI₂(L) (pyridine).

Iridium Complexes:

The iridium complex can be obtained according to the method described in the literature (K. Mashima et al., J. Organomet. Chem., 1992, 428, 213) and other literatures. More specifically, the iridium complex can be obtained by reacting a chiral ligand with [Ir(cod) (CH₃CN)₂]BF₄ in tetrahydrofuran with stirring.

Specific examples of the iridium complexes include, for example, those given below: [Ir(L)Cl]₂, [Ir(L)Br]₂, [Ir(L)I]₂, [Ir(cod)(L)]BF₄, [Ir(cod)(L)]ClO₄, (Ir(cod)(L)]PF₆, [Ir(cod)(L)]BPh₄, [Ir(cod)(L)]OTf, [Ir(nbd)(L)]BF₄, [Ir(nbd)(L)]ClO₄, [Ir(nbd)(L)]PF₆, [Ir(nbd)(L)]BPh₄ and [Ir(nbd)(L)]OTf.

Palladium Complexes:

The palladium complex can be obtained according to the method described in the literatures (Y. Uozumi et al., J. Am. Chem. Soc., 1991, 9887, etc.). More specifically, they can be obtained by reacting a chiral ligand with n-allylpalladium chloride.

Specific examples of the palladium complex include, for example, those which follow: PdCl₂(L), (n-allyl)Pd(L), [Pd(L)]BF₄, [Pd(L)]ClO₄, [Pd(L)]PF₆, [Pd(L)]BPh₄ and [Pd(L)]OTf.

Nickel Complexes:

The nickel complex can be obtained according to the method described in “JIKKEN KAGAKU KOZA, 4^{th} Ed., Volume 18, Organic Metal Complexes, p. 376, published by Maruzen, in 1991” and in other literatures. The nickel complex can also be obtained, according to the method described in the literature (Y. Uozumi et al., J. Am. Chem. Soc., 1991, 113, 9887), by dissolving a chiral ligand and nickel chloride in a mixture of 2-propanol and methanol and heating the resultant solution with stirring.

Specific examples of the nickel complex include, for example, those which follow: NiCl₂(L), NiBr₂(L) and NiI₂(L).

As the transition metal complexes, both commercially available products and those synthesized in-house can be used.

These transition metal complexes can be obtained by reacting the chiral ligand with a transition metal compound. In the case of using the complex as the catalyst, the transition metal complex may be used after increasing its purity or the obtained transition metal complex may be used without purification i.e. in situ.

The transition metal compound represented by the following formula: [MXₘLₙ]ₚ

wherein M, X, L, m, n and p are each the same meaning as defined above.

As the above formula, concrete examples of Ru, Rh and Ir are exemplified. Specific examples of the above formula include, for example, [RuCl₂(benzene)]₂, [RuBr₂(benzene)]₂, [RuI₂(benzene)]₂, [RuCl₂(p-cymene)]₂, [RuBr₂(p-cymene)]₂, [RuI₂(p-cymene)]₂, RuCl₂(hexamethylbenzene)]₂, [RuBr₂(hexamethylbenzene)]₂, [RuI₂(hexamethylbenzene)]₂, [RuCl₂(mesitylene)]₂, [RuBr₂(mesitylene)]₂, [RuI₂(mesitylene)]₂, [RuCl₂(pentamethylcyclopentadiene)]₂, [RuBr₂(pentamethylcyclopentadiene)]₂, [RuI₂(pentamethylcyclopentadiene)]₂, [RuCl₂(cod)]₂, [RuBr₂(cod)]₂, [RuI₂(cod)]₂, [RuCl₂(nbd)₂, [RuBr₂(nbd)]₂, [RuI₂(nbd)]₂, RuCl₃ hydrate, RuBr₃ hydrate, RuI₃ hydrate, [RhCl₂(cyclopentadiene)]₂, [RhBr₂(cyclopentadiene)]₂, [RhI₂(cyclopentadiene)]₂, [RhCl₂(pentamethylcyclopentadiene)]₂, [RhBr₂(pentamethylcyclopentadiene)]₂, [RhI₂(pentamethylcyclopentadiene)]₂, [RhCl(cod)]₂, [RhBr(cod)]₂, [RhI(cod)]₂, [RhCl(nbd)]₂, [RhBr(nbd)₂, [RhI(nbd)]₂, RhCl₃ hydrate, RhBr₃ hydrate, RhI₃ hydrate, [IrCl₂(cyclopentadiene)]₂, [IrBr₂(cyclopentadiene)]₂, [IrI₂(cyclopentadiene)]₂, [IrCl₂(pentamethylcyclopentadiene)]₂, [IrBr₂(pentamethylcyclopentadiene)]₂, [IrI₂(pentamethylcyclopentadiene)]₂, [IrCl(cod)]₂, [IrBr (cod)]₂, [IrI(cod)]₂, [IrCl(nbd)]₂, [IrBr(nbd)]₂, (IrI(nbd)]₂, IrCl₃ hydrate, IrBr₃ hydrate, and IrI₃ hydrate.

Among the transition metal complexes which can be used in the present invention, those which have chiral ligands are preferably used, and, furthermore, those which have chiral phosphine ligands as the chiral ligands mentioned above are used more preferably.

Although the amount of the chiral catalyst used depends on the above-mentioned cinnamic acid (4), the reaction vessel used, the reaction mode and the production cost, it is usually selected appropriately from the range of 1/10 to to 1/100,000 in mole or preferably from the range of 1/50 to 1/10,000 in mole to the cinnamic acid (4).

The hydrogen pressure in the process of the present invention is sufficient in such a condition of hydrogen atmosphere or 0.1 MPa, however, it is usually selected appropriately from the range of 0.1 to 20 MPa, preferably 0.2 to 10 MPa in view of economical cost. Further, it is possible to maintain high activity even at a pressure of not higher than 1 MPa in view of economical cost.

The asymmetric hydrogenation is carried out optionally in the presence of a solvent. The solvent includes, for example, aromatic hydrocarbons such as benzene, toluene, xylene, etc. , aliphatic hydrocarbons such as pentane, hexane, heptane, octane, etc., halogenated hydrocarbons such as dichloromethane, chloroform, carbon tetrachloride, dichloroethane, etc., ethers such as diethyl ether, diisopropyl ether, tert-butyl methyl ether, dimethoxyethane, tetrahydrofuran, dioxane, dioxolane, etc., alcohols such as methanol, ethanol, 2-propanol, n-butanol, tert-butanol, benzyl alcohol, etc., polyalcohols such as ethylene glycol, propylene glycol, 1,2-propanediol, glycerin, etc., amides such as N,N-dimethylformamide, N,N-dimethylacetamide, etc., acetonitrile, N-methylpyrrolidone, dimethyl sulfoxide, water, etc. These solvents may be used solely or appropriately in combination with two or more kinds of solvents.

The amount of the solvent used can be determined in view of solubility and economical cost of the cinnamic acid (4) which is a reaction substrate. For example, when an alcohol is used as a solvent, it is possible to carry out the reaction at a concentration of from not more than 1% to in the absence of a solvent or in the almost absence of a solvent, depending on the cinnamic acid (4). Usually, the concentration of the cinnamic acid (4) is selected appropriately from the range of 5 to 50% by mass, preferably 10 to 40% by mass.

Usually, the reaction temperature is selected appropriately from the range of 15 to 100° C., preferably 20 to 80° C. in view of economical cost. Further, it is possible to carry out the reaction even at a low temperature of −30 to 0° C. or a high temperature of 100 to 250° C.

The reaction is complete within several minutes to several hours, though it varies with the reaction conditions such as the kinds and amounts of the chiral hydrogenation catalysts used, the kinds and concentrations of the cinnamic acid (4), the reaction temperature, and the hydrogen pressure. Usually, the reaction time is selected appropriately from the range of one minute to 48 hours, preferably 10 minutes to 24 hours,

The asymmetric hydrogenation of the present invention may be carried out by a batch-method or a continuous method.

The optically active phenylpropionic acids (5) obtained in the above-mentioned process may be, if necessary, converted into optically active phenylpropionic acids with optically higher purity and/or chemically higher purity or salts thereof by various procedures.

Such various procedures include, for example, crystallization, column chromatography and the like.

The crystallization may be carried out by the conventional method used in this field.

Examples of the solvent used in the crystallization include hydrocarbons such as aromatic hydrocarbons such as benzene, toluene, xylene, etc. and aliphatic hydrocarbons such as pentane, hexane, heptane, octane, etc.; halogenated hydrocarbons such as dichloromethane, chloroform, carbon tetrachloride, dichloroethane, etc.; ethers such as diethyl ether, diisopropyl ether, tert-butyl methyl ether, Idimethoxyethane, tetrahydrofuran, dioxane, dioxolane, etc.; alcohols such as methanol, ethanol, 2-propanol, n-butanol, tert-butanol, benzyl alcohol, etc.; polyalcohols such as ethylene glycol, propylene glycol, 1,2-propanediol, glycerin, etc.; amides such as N,N-dimethylformamide, formamide, N,N-dimethylacetamide, etc.; ketones such as acetone, methyl ethyl ketone, methyl isobutyl ketone, cyclohexanone, etc.;

acetonitrile, N-methylpyrrolidone, dimethyl sulfoxide, water, or the like. These solvents may be used alone or appropriately in combination of two or more of them. The hydrocarbon solvents such as aromatic hydrocarbons and aliphatic hydrocarbons; alcohols; ketones; water; etc. and a mixture thereof are preferable.

As used herein, “optically higher purity” means a higher optical purity, substantially 100% ee, than optical purities of optically active phenylpropionic acids (5), or optically active 3-(4-hydroxyphenyl)propionic acids (6) obtained in the above-mentioned processes. Here, the “substantially 100% ee” means an optical purity where one mirror image over the other mirror image is almost not detectable. In the present invention, such substantially 100% ee is specifically an optical purity of ≧95% ee, preferably ≧97% ee, more preferably ≧98% ee, still more preferably ≧99% ee.

Also, “chemically higher purity” means a higher chemical purity, substantially 100%, than chemical purities of optically active phenylpropionic acids (5), or optically active 3-(4-hydroxyphenyl)propionic acids (6) obtained in the above-mentioned process. Here, the “substantially 100%” means a chemical purity where any other compounds are almost not detectable. In the present invention, such substantially 100% is specifically a chemical purity of ≧95%, preferably ≧97%, more preferably ≧98%, still more preferably ≧99%.

The optically active phenylpropionic acid (5) obtained in the above asymmetric hydrogenation is deprotected to give the desired optically active 3-(4-hydroxyphenyl)propionic acid (6).

The deprotection is carried out by conventional methods.

For example, such deprotection may be conducted according to the methods described in “PROTECTIVE GROUPS IN ORGANIC SYNTHESIS THIRD EDITION (JOHN WILEY & SONS, INC. (1999))”, “Basic Knowledge and Experiment in Peptide Synthesis, published by Maruzen in 1985” or “JIKKEN KAGAKU KOZA, 4^{th} Ed., Volume 18, Organic Metal Complexes, pp. 339-344, published by Maruzen, in 1991”. To be specific, when the protective group is a benzyl group, such protective group is removed by catalytic hydrogenation using a palladium-carbon catalyst.

The reaction temperature is usually selected appropriately from the range of 0° C. to the boiling point of the solvent used, preferably 20° C. to 80° C.

The reaction time is usually selected appropriately from the range of 0.1 to 48 hours, preferably 1 to 10 hours.

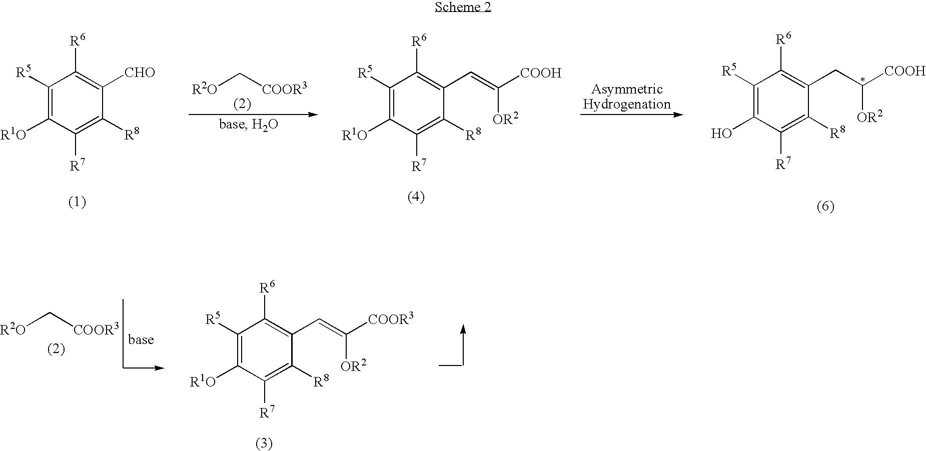

The optically active 3-(4-hydroxyphenyl)propionic acid (6) can be produced by preparing a cinnamic acid (4) in accordance with the above scheme 1 and subjecting the resulting cinnamic acid (4) to an asymmetric hydrogenation.

This method of scheme 2 is able to simultaneously carry out an asymmetric hydrogenation and a deprotection.

The kinds and amounts of the asymmetric hydrogenation catalysts, and the reaction conditions used in the present invention are each the same as those described in the above

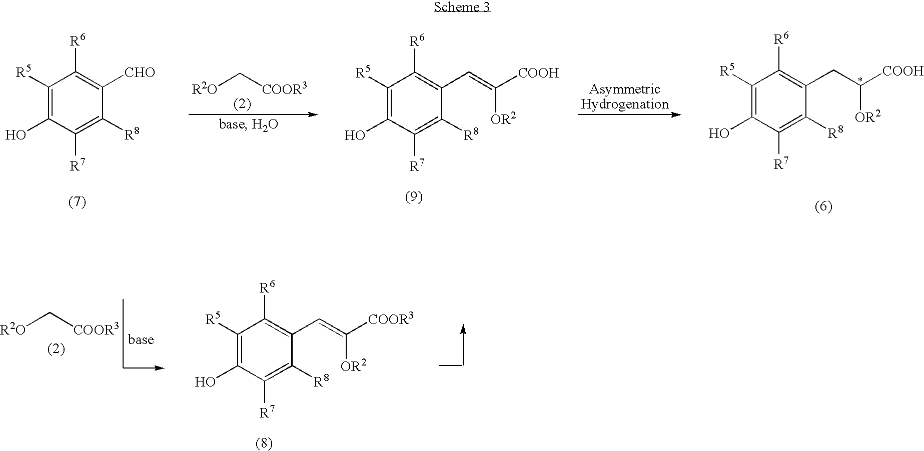

The 4-hydroxycinnamic acid (9) can be produced by reacting a 4-hydroxybenzaldehyde (7) with a glycolic acid derivative (2) in a suitable solvent in the presence of a base, followed by hydrolysis.

The solvents, bases and the reaction conditions such as reaction temperature and reaction time are each the same as those described in the above-mentioned scheme 1.

The amount of the solvent used is usually 0.1- to 100-fold amount, preferably 1- to 20-fold amount of the 4-hydroxybenzaldehyde (7).

The amount of the base used is usually selected appropriately from 0.0 1- to 10-fold amount of the glycolic acid derivative (2), preferably 1- to 5-fold amount of the glycolic acid derivative (2).

The 4-hydroxycinnamic acid (9) may be produced by reacting a 4-hydroxybenzaldehyde (7) with a glycolic acid derivative (2), if required, subjecting the product to post-treatment and purification, and isolating the 4-hydroxycinnamic acid ester of the formula (8): 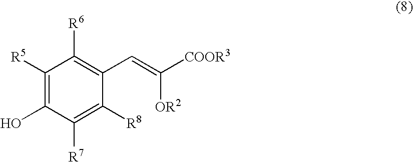
wherein R², R³, and R⁵ to R⁸ are each the same as defined above (hereinafter, if required, called as 4-hydroxycinnamic acid ester (8)), followed by hydrolysis, thereby giving a cinnamic acid (9). Alternatively, without isolation of the cinnamic acid ester (8), hydrolysis may be carried out upon addition of water alcohol and/or the above mentioned base.

Specific examples of the 4-hydroxycinnamic acid ester (8) include methyl 3-(4-hydroxyphenyl)-2-methoxyacrylate, ethyl 3-(4-hydroxyphenylZ)-2-methoxyacrylate, propyl 3-(4-hydroxyphenyl)-2-methoxyacrylate, butyl 3-(4-hydroxyphenyl)-2-methoxyacrylate, tert-butyl 3-(4-hydroxyphenyl)-2-methoxyacrylate, methyl 3-(4-hydroxyphenyl)-2-ethoxyacrylate. ethyl 3-(4-hydroxyphenyl)-2-ethoxyacrylate, propyl 3-(4-hydroxyphenyl)-2-ethoxyacrylate, butyl 3-(4-hydroxyphenyl)-2-ethoxyacrylate, tert-butyl 3-(4-hydroxyphenyl)-2-ethoxyacrylate, methyl 3-(4-hydroxyphenyl)-2-propoxyacrylate, ethyl 3-(4-hydroxyphenyl)-2-propoxyacrylate, propyl 3-(4-hydroxyphenyl)-2-propoxyacrylate, butyl 3-(4-hydroxyphenyl)-2-propoxyacrylate, tert-butyl 3-(4-hydroxyphenyl)-2-propoxyacrylate, methyl 3-(4-hydroxyphenyl)-2-butoxyacrylate, ethyl 3-(4-hydroxyphenyl)-2-butoxyacrylate, ropyl 3-(4-hydroxyphenyl)-2-butoxyacrylate, butyl 3-(4-hydroxyphenyl)-2-butoxyacrylate, tert-butyl 3-(4-hydroxyphenyl)-2-butoxyacrylate, etc.

The resulting cinnamic acid (9) may be a mixture of a cinnamic acid of the formula (9) wherein the carboxy group is free, and a metal salt of a cinnamic acid of the formula (9-1) and/or a cinnamic acid amine salt of the formula (9-2).

Further, the cinnamic acid (9) thus obtained is, if required, converted into a metal salt of a cinnamic acid of the formula (9-1) or a cinnamic acid amine salt of the formula (9-2), or a salt different from a salt of the cinnamic acid of the formula (9), using the above-mentioned base.

The desired optically active 3-(4-hydroxyphenyl)propionic acid (6) can be produced by subjecting the obtained 4-hydroxycinnamic acid (9) to asymmetric hydrogenation.

The asymmetric hydrogenation may be carried out in accordance with the procedure as shown in the above scheme 1.

The amount of the chiral catalyst used is usually selected from the range of molar ratio of 1/10 to 1/100,000, preferably 1/50 to 1/10,000, to the 4-hydroxycinnamic acid (9), though it varies with the reaction vessel, the reaction mode and economical cost.

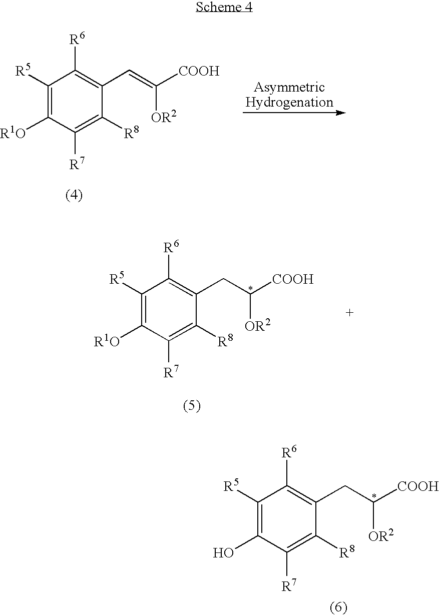

Scheme 4 illustrates the reaction wherein the cinnamic acid (4) obtained as shown in scheme 1 is subjected to asymmetric hydrogenation to give a mixture of an optically active phenylpropionic acid (5) and an optically active 3-(4-hydroxyphenyl)propionic acid (6). In this reaction, (i) the resultant mixture may be in situ deprotected to give the desired optically active 3-(4-hydroxyphenyl)propionic acid (6), or (ii) the optically active phenylpropionic acid (5) and the optically active 3-(4-hydroxyphenyl)propionic acid (6) may be separated respectively and the separated optically active phenylpropionic acid (5) may be deprotected to give the desired optically active 3-(4-hydroxyphenyl)propionic acid (6).

Thus obtained optically active 3-(4-hydroxyphenyl)propionic acid (6) may be subjected to post-treatment, if required.

Furthermore, the optically active 3-(4-hydroxyphenyl)propionic acids (6) obtained in the above-mentioned process may be, if necessary, converted into optically active 3-(4-hydroxyphenyl)propionic acids (6) with optically higher purity and/or chemically higher purity by various procedures.

Such various procedures include, for example, crystallization, column chromatography and the like.

The crystallization is illustrated as in the above scheme 1.

As used herein, “optically higher purity” means a higher optical purity, substantially 100% ee, than optical purities of optically active 3-(4-hydroxyphenyl)propionic acids (6) obtained in the above-mentioned process. Here, the “substantially 100% ee” means an optical purity where one mirror image over the other mirror image is almost not detectable. In the present invention, such substantially 100% ee is specifically an optical purity of ≧95% ee, preferably ≧97% ee, more preferably ≧98% ee, still more preferably ≧99% ee.

Also, “chemically higher purity” means a higher chemical purity, substantially 100%, than chemical purities of optically active 3-(4-hydroxyphenyl)propionic acids (6) obtained in the above-mentioned process. Here, the “substantially 100%” means a chemical purity where any other compounds are almost not detectable. In the present invention, such substantially 100% is specifically a chemical purity of ≧95%, preferably ≧97%, more preferably ≧98%, still more preferably ≧99%.

The resulting optically active 3-(4-hydroxyphenyl)propionic acid of the formula (6) or a salt thereof may be a mixture of the propionic acid of the formula (6) wherein the carboxy group is free, and a metal salt of an optically active 3-(4-hydroxyphenyl)propionic acid of the formula (6-1) and/or an optically active 3-(4-hydroxyphenyl)propionic acid amine salt of the formula (6-2).

Further, the resulting optically active 3-(4-hydroxyphenyl)propionic acid of the formula (6) may be, if required, converted into a metal salt of an optically active 3-(4-hydroxyphenyl)propionic acid of the formula (6-1), or an optically active 3-(4-hydroxyphenyl)propionic acid amine salt of the formula (6-2), or a salt different from the salt of the 3-(4-hydroxyphenyl)propionic acid, using the above-mentioned base.

Thus obtained optically active 3-(4-hydroxyphenyl)propionic acid (6) is useful as intermediates for medicines and the like.

The production of an optically active α,β-unsaturated carboxylic acid of the formula (12): 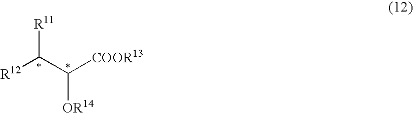
wherein R¹¹ to R¹² are each independently a hydrogen atom or a substituent; R¹³ is a hydrogen atom, an optionally substituted hydrocarbon group or a metal salt; R¹⁴ is a hydrogen atom or a protective group; and * is an chiral carbon atom, or a salt thereof can be produced by subjecting an optically active α,β-unsaturated carboxylic acid of the formula (11): 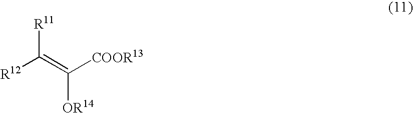
wherein R¹¹ to R¹⁴ are each the same as defined above, or a salt thereof, to asymmetric hydrogenation.

As the substituent represented by R¹¹ and R¹² in the formula (12), there are exemplified an optionally substituted hydrocarbon group, an optionally substituted heterocyclic group, an optionally substituted alkoxy group, an optionally substituted aralkyloxy group, an optionally substituted aryloxy group, an optionally substituted alkoxycarbonyl group, an optionally substituted aryloxycarbonyl group, and an optionally substituted aralkyloxycarbonyl group.

The optionally substituted hydrocarbon group includes a hydrocarbon group and a substituted hydrocarbon group. Such hydrocarbon group includes, for example, alkyl, alkenyl, alkynyl, aryl and aralkyl.

The alkyl, aryl, and aralkyl groups may be each the same meaning as each group described for the protective group represented by R¹ in the production of the optically active 3-(4-hydroxyphenyl)propionic acid of the formula (6) or a salt thereof.

The alkenyl group may be linear or branched, and includes an alkenyl group of 2 to 20 carbon atoms, preferably 2 to 10 carbon atoms, more preferably 2 to 6 carbon atoms. Specific examples of such alkenyl group are ethenyl, propenyl, 1-butenyl, pentenyl, hexenyl, etc.

The alkynyl group may be linear or branched, and includes, for example, an alkynyl group of 2 to 20 carbon atoms, preferably 2 to 10 carbon atoms, more preferably 2 to 6 carbon atoms. Specific examples of such alkynyl group are ethynyl, 1-propynyl, 2-propynyl, 1-butynyl, 3-butynyl, pentynyl, hexynyl, etc.

The substituted hydrocarbon group (hydrocarbon group having a substituent) is a hydrocarbon group formed by substituting one hydrogen atom of the above-mentioned hydrocarbon group by a substituent. The substituted hydrocarbon group includes a substituted alkyl group, a substituted alkenyl group, a substituted alkynyl group, a substituted aryl group, a substituted aralkyl group, etc. The substituent will be described hereinafter.

The optionally heterocyclic group includes a heterocyclic group and a substituted heterocyclic group. As the heterocyclic group, there are exemplified an aliphatic heterocyclic group and an aromatic heterocyclic group. The heterocyclic group and the substituted heterocyclic group are each the same as each group defined for the protective group represented by R¹ in the production of optically active 3-(4-hydroxyphenyl)propionic acids or salts thereof.

The substituted heterocyclic group (heterocycic group having a substituent) is a heterocyclic group wherein at least one hydrogen atom of the above-mentioned heterocyclic group is substituted by a substituent. The substituted heterocyclic group (heterocycic group having a substituent) includes a substituted aliphatic heterocyclic group and a substituted aromatic heterocyclic group. The substituent will be described hereinafter.

The optionally substituted alkoxy group:includes an alkoxy group and a substituted alkoxy group.

The optionally substituted aralkyloxy group includes an aralkyloxy group and a substituted aralkyloxy group.

The optionally substituted aryloxy group includes an aryloxy group and a substituted aryloxy group.

The optionally substituted alkoxycarbonyl group is an alkoxycarbonyl group and a substituted alkoxycarbonyl group.

The substituted aryloxycarbonyl group is an aryloxycarbonyl group and a substituted aryloxycarbonyl group.

The optionally substituted aralkyloxycarbonyl group includes an aralkyloxycarbonyl group and a substituted aralkyloxycarbonyl group.

These alkoxy group, substituted alkoxy group, aralkyloxy group, substituted aralkyloxy group, aryloxy group, substituted aryloxy group, alkoxycarbonyl group, substituted alkoxycarbonyl group, aryloxycarbonyl group, substituted aryloxycarbonyl group, aralkyloxycarbonyl group, and substituted aralkyloxycarbonyl group are each the same as each group described for the protective group represented by R¹ in the production of optically active 3-(4-hydroxyphenyl)propionic acids of the above formula (6) or salts thereof.

As the substituent, there are exemplified a hydrocarbon group, a substituted hydrocarbon group, a halogen atom, a halogenated hydrocarbon group, a heterocyclic group, a substituted heterocyclic group, an alkoxy group, a substituted alkoxy group, an aralkyloxy group, a substituted aralkyloxy group, an aryloxy group, a substituted aryloxy group, an alkylthio group, a substituted alkylthio group, an arylthio group, a substituted arylthio group, an aralkylthio group, a substituted aralkylthio group, an acyl group, a substituted acyl group, an acyloxy group, a substituted acyloxy group, an alkoxycarbonyl group, a substituted alkoxycarbonyl group, an aryloxycarbonyl group, a substituted aryloxycarbonyl group, an aralkyloxycarbonyl group, a substituted aralkyloxycarbonyl group, an alkylenedioxy group, a hydroxy group, a nitro group, an amino group, a substituted amino group, a cyano group, a carboxy group, a sulfo group, a sulfonyl group, a substituted silyl group, etc.

These substituents may be the same as those mentioned in the production of optically active 3-(4-hydroxyphenyl)propionic acids (6).

The optionally substituted hydrocarbon group represented by R¹³ may be the same as the hydrocarbon group mentioned for the above R¹¹, and R¹².

The metal atom includes an alkali metal and an alkaline earth metal.

The alkali metal and alkaline earth metal may be the same as the alkali metal and alkaline earth metal mentioned for the above formula (4-1).

The protective group represented by R¹⁴ may have the same meaning as defined for the protective group represented by R¹ in the production of optically active 3-(4-hydroxyphenyl)propionic acids (6).

In the case where R¹¹ and/or R¹² in the formula (12) are a hydrogen atom, the carbon atom to which R¹¹ and R¹² are attached cannot be an chiral carbon atom. Further, when R¹¹ and R¹² are the same each other, the carbon atom to which R¹¹ and R¹² are attached cannot be an chiral carbon atom.

As the salt of α,β-unsaturated carboxylic acid, there are exemplified a salt of an α,β-unsaturated carboxylic acid wherein R¹³ in the formula (11) is a metal atom such as an alkali metal and alkaline earth metal, and an α,β-unsaturated carboxylic acid amine salt of the formula (11-1): 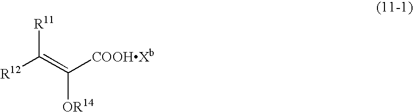
wherein X^{b} is an amine; R¹¹, R¹²and R¹⁴ are each the same as defined above.

The amine represented by X^{b} is the same as that defined for X^{a} in the above formula (4-2).

As the examples of α,β-unsaturated carboxylic acids of the formula (11) and α,β-unsaturated carboxylic acid salts of the formula (11-1), there are exemplified examples of the cinnamic acid of the formula (4), examples of the cinnamic acid salt of the above formulae (4-1) and (4-2), examples of the 4-hydroxycinnamic acid of the formula (9) and examples of the 4-hydroxycinnamic acid salt of the formulae (9-1) and (9-2), and following compounds:

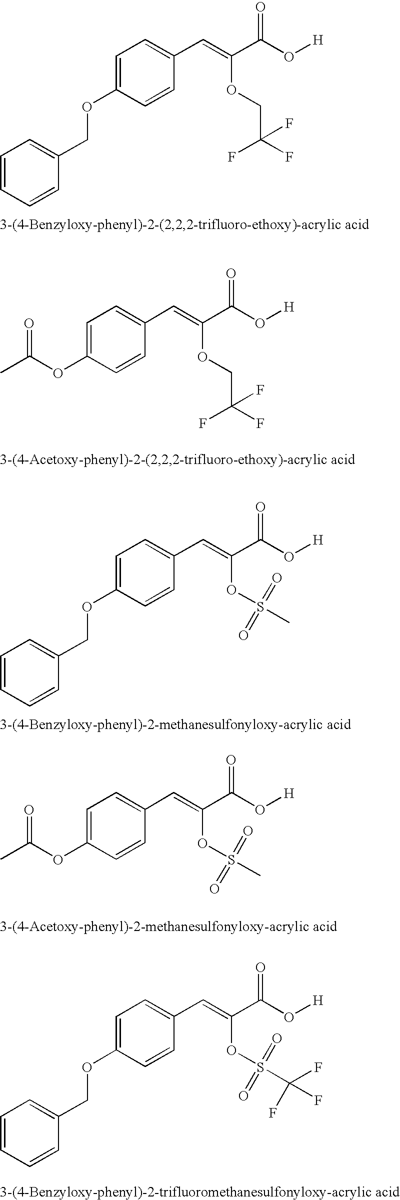

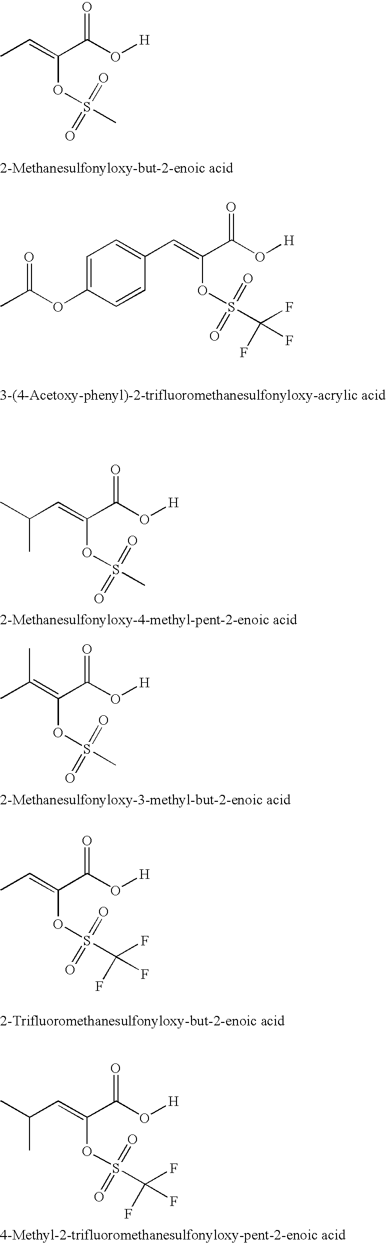

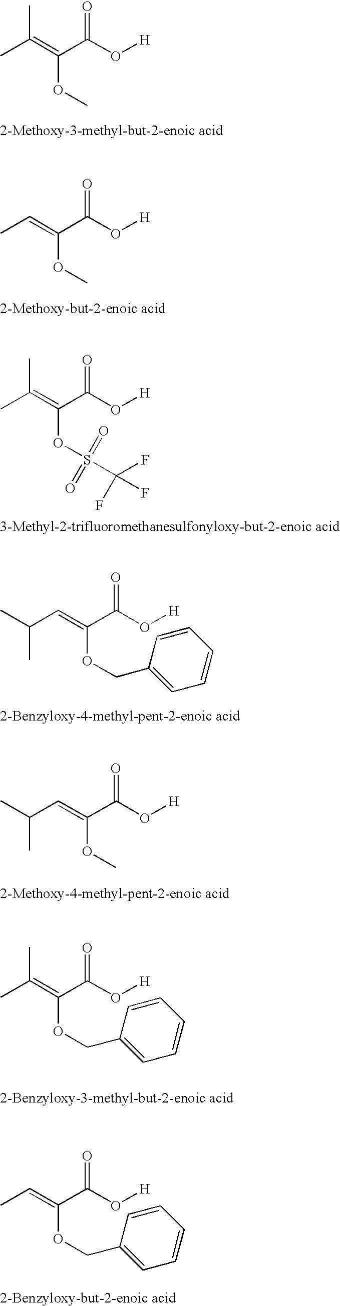

Further, as the optically active carboxylic acid salt, there are exemplified a salt of an optically active carboxylic acid of the formula (12) wherein R¹³ is a metal atom such as an alkali metal and an alkaline earth metal, and an optically active carboxylic acid amine salt of the formula (12-1): 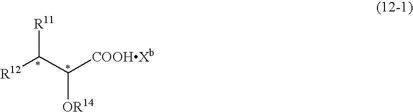
wherein R¹¹, R¹², R¹⁴, X^{b} and * are each the same as defined above.

As the examples of optically active carboxylic acid of the formula (12) and salts of optically active carboxylic acid of the formula (12-1) obtained in accordance with the present invention, there are exemplified compounds such as optically active 3-(4-hydroxyphenyl)propionic acids of the above formula (6), salts of optically active 3-(4-hydroxyphenyl)propionic acids of the above formula (6-1), and optically active 3-(4-hydroxyphenyl)propionic acid salts of the formula (6-2), and following compounds:

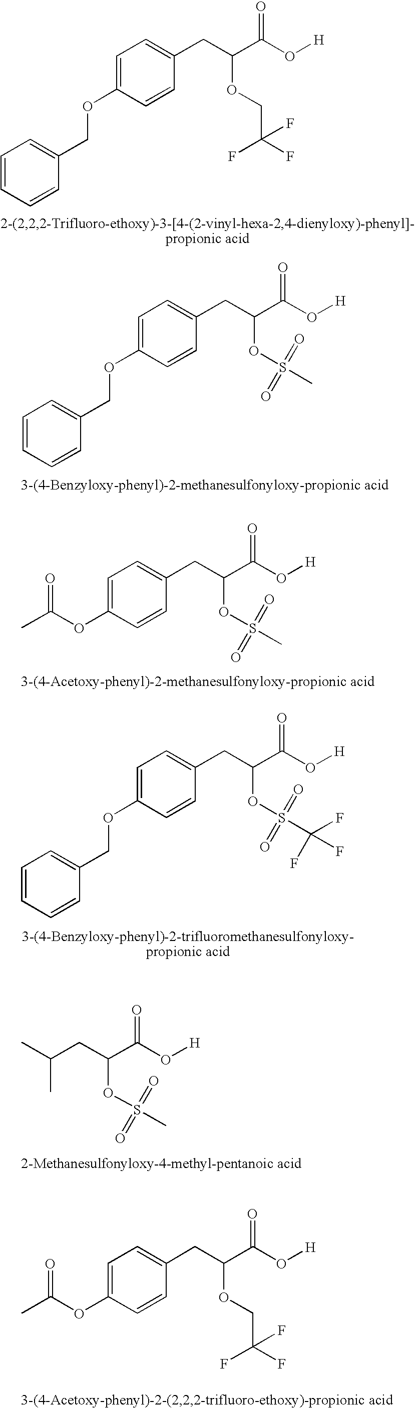

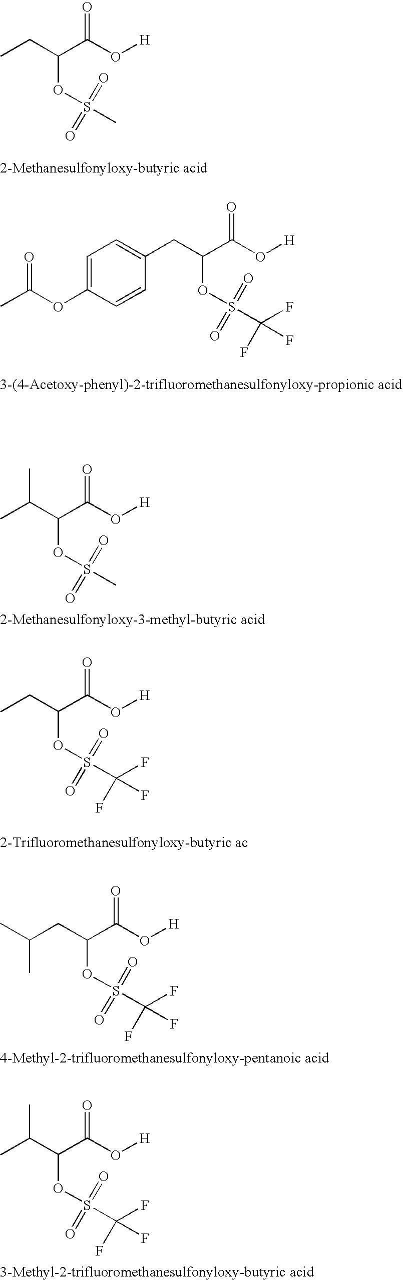

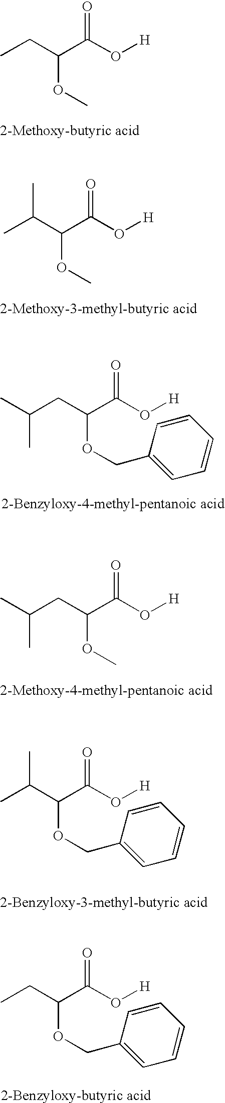

The asymmetric hydrogenation is carried out in the presence of a chiral catalyst. The chiral catalyst and ther reaction conditions are the same as those described in the production of the above optically active 3-(4-hydroxyphenyl)propionic acids (6). In the case that the transition metal is rhodium, the protective group represented by R¹⁴ in the above formula (11) is a group other than acyl groups.

Usually, the amount of the chiral catalyst used is selected appropriately from the range of 1/10 to 1/100,000, preferably 1/50 to 1/10,000, in a molar ratio to the α,β-unsaturated carboxylic acid or salt thereof, though it varies with the α,β-unsaturated carboxylic acid of the formula (11) or salt thereof, the reaction vessel, the reaction mode and economical cost.

Thus obtained optically active carboxylic acids of the formula (12) or salts thereof may be a mixture of an optically active carboxylic acid wherein the carboxy group is free (R¹³ is a hydrogen atom), a salt of an optically active carboxylic acid of the above formula (12) wherein R¹³ is a metal atom, and an optically active carboxylic acid amine salt of the above formula (12-1).

Further, the optically active carboxylic acid of the formula (12) thus obtained is, if required, converted into a metal salt of an optically active carboxylic acid of the formula (12), an optically active carboxylic acid amine salt of the formula (12-1), or a salt different from a salt of an optically active carboxylic acid of the formula (12), using the above-mentioned base.

Thus obtained optically active carboxylic acids of the formula (12) or salts thereof are useful as intermediates for medicines, etc.

### EXAMPLES

The present invention is illustrated in more detail by referring to the following Examples and Reference Examples. However, the present invention is in no way restricted by these Examples.

Chemical purity and enantiomeric excess were determined by high performance liquid chromatography.

¹H-NMR was determined by using Varian GEMINI-2000 (200 MHz).

### Example 1

### Synthesis of methyl 3-(4-benzyloxyphenyl)-2-methoxyacrylate

To a mixture of benzyloxybenzaldehyde (21.24 g, 100 mmol), sodium methoxide (18.77 g, 330 mmol) and methanol (200 mL) was added methyl methoxyacrylate (30.00 g, 297 mmol) in a nitrogen stream, and the mixture was heated under ref lux for 5 hours. The reaction mixture was concentrated and diluted with butyl acetate. The organic layer was washed, and concentrated. The residue was purified by column chromatography on silica gel to give the title compound (23.8 g) in 80% yield.
¹H-NMR δ (CDCl₃): 3.76 (3H, s), 3.85 (3H, s), 5.10 (2H, s), 6.97 (1H, s), 6.98 (2H, d, J=8.8 Hz), 7.30-7.50 (5H, m), 7.72 (2H, d, J=8.8 Hz).

### Example 2

### Synthesis of sodium 3-(4-benzyloxyphenyl)-2-methoxyacrylate

To a mixture of methyl 3-(4-benzyloxyphenyl)-2-methoxyacrylate (20 g, 67.0 mmol) and methanol (200 mL) was added 1N sodium hydroxide (74 mL), and the mixture was heated under ref lux for 2 hours. The reaction mixture was cooled down to room temperature, and the resulting precipitates were collected by filtration to give the title compound (17.44 g) in 85% yield.
¹H-NMR δ (CD₃OD): 3.69 (3H, s), 5.08 (2H, s), 6.63 (1H, s), 6.93 (2H, d, J=9.0 Hz), 7.25-7.50 (5H, m), 7.64 (2H, d, J=9.0 Hz).

### Example 3

### Synthesis of sodium 3-(4-benzyloxyphenyl)-2-methoxyacrylate

To a mixture of benzyloxybenzaldehyde (21.24 g, 100 mmol), sodium methoxide (18.77 g, 330 mmol) and methanol (200 mL) was added methyl methoxyacetate (30.00 g, 297 mmol) in a nitrogen stream, and the mixture was heated under reflux for 5 hours. After addition of water (40 mL), the mixture was heated under ref lux for 1.5 hours, and cooled down to room temperature. The resulting precipitates were collected by filtration to give the title compound (20.02 g) in 65% yield.

### Example 4

### Synthesis of sodium 3-(4-benzyloxyphenyl)-2-methoxypropionate

Sodium 3-(4-benzyloxyphenyl)-2-methoxyacrylate (19.65 g, 64.15 mmol), Ru₂Cl₄[(S)—H₈-binap]₂NEt₃ (57.5 mg) and methanol (200 mL) were placed in a 200 ml-autoclave, and hydrogen gas was supplied to a required pressure of 5 MPa. The mixture was stirred at 60° C. for 6.5 hours, and the solvent was removed by evaporation in vacuo to give sodium 3-(4-benzyloxyphenyl)-2-methoxypropionate (19.7 g, 90% ee) ¹H-NMR δ (CD₃OD): 2.78 (1H, dd, J=14.4, 8.8 Hz), 2.94 (1H, dd, 14.4, 4.0 Hz), 3.23 (3H, s), 3.69 (1H, dd, J=8.8, 4.0 Hz), 5.03 (2H, s), 6.86 (2H, d, J=8.8 Hz), 7.1764 (2H, d, J=8.8 Hz), 7.25-7.46 (5H, m).

### Example 5

### Synthesis of sodium 3-(4-hydroxyphenyl)-2-methoxypropionate

Sodium 3-(4-benzyloxyphenyl)-2-methoxyacrylate (250 mg, 0.816 mmol) and [Ru(p-cymene)((S)-dm-segphos)]Cl (4.2 mg, 0.0041 mmol) were placed in a 100 ml-autoclave, and the atmosphere in the reaction system was substituted by nitrogen. After addition of methanol (2.5 mL), hydrogen gas (5.0 MPa) was introduced thereto, and the mixture was stirred at 60° C. for 16 hours. After the reaction, the reactant was a mixture of sodium 3-(4-hydroxyphenyl)-2-methoxypropionate and sodium 3-(4-benzyloxyphenyl)-2-methoxypropionate. The ratio of the sodium 3-(4-hydroxyphenyl)-2-methoxypropionate/sodium 3-(4-benzyloxyphenyl)-2-methoxypropionate was found to be 16/84 by means of ¹H-NMR. The reaction product was purified and isolated to give the title compound (36 mg) in 20% yield with 92.9% ee.

### Example 6

### Synthesis of 3-(4-hydroxyphenyl)-2-methoxyacrylic acid

Methanol (200 mL) was added to a mixture of 4-hydroxybenzaldehyde (20.5 g, 168 mmol) and sodium methoxide (36.3 g, 672 mmol). Then, methyl methoxyacetate (50 mL, 504 mmol) was added dropwise to the above mixture at 50° C. to 60° C.

The resulting mixture was heated under ref lux for 12 hours, and water (40 mL) was added. The mixture was further stirred for 2 hours under reflux, cooled down to room temperature, and concentrated in vacuo to remove the solvent. To the residue were added 1N hydrochloric acid and dichloromethane, and the resulting solid was collected by filtration. The solid was washed with water and dried to give the title compound (20.3 g) in 62% yield.
m.p. 163-165° C.
¹H NMR δ (CD₃OD): 7.65 (d, J=8.4 Hz, 2H), 6.99 (s, 1H), 6.81 (d, J=8.4 Hz, 2H), 3.74 (s, 3H).

### Example 7

### Synthesis of methyl 3-(4-hydroxyphenyl)-2-methoxyacrylate

To a mixture of 4-hydroxybenzaldehyde (1.0 g, 8.19 mmol) and sodium methoxide (1.77 g, 32.8 mmol) were added toluene (5 mL) and methanol (10 mL) in a nitrogen stream. After addition of methyl methoxcyacetate (2.44 mL, 24.6 mmol), the mixture was stirred at room temperature for one hour, then heated under reflux for 8 hours. The reaction mixture was cooled down to room temperature and saturated aqueous ammonium chloride (40 mL) was added. The mixture was extracted twice with ethyl acetate (40 mL), and the organic layer was washed with saturated brine (40 mL), dried over sodium sulfate and concentrated in vacuo to remove the solvent. The resulting crude product was purified by column chromatography on silica gel to give the title compound (1.51 g) of 74% purity in 89% yield.
¹H NMR δ (CDCl₃): 7.67 (d, J=8.6 Hz, 2H), 6.97 (s, 1H), 6.85 (d, J=8.6 Hz, 2H), 5.68 (s, 1H), 3.85 (s, 3H), 3.75 (s, 3H).

### Example 8

### Synthesis of 3-(4-hydroxyphenyl)-2-methoxyacrylic acid

Methyl 3-(4-hydroxyphenyl)-2-methoxyacrylate (1.51 g) prepared in Example 7 was dissolved in methanol (10 mL), and 1N sodium hydroxide (7.8 mL) was added thereto. The solution was heated under reflux for 2 hours, cooled down to room temperature and concentrated in vacuo to remove the solvent. After addition of 1N hydrochloric acid and dichloromethane to the residue, the resulting solid was collected by filtration, washed with water and dried to give the title compound (857 mg). The ¹H NMR spectrum was identical with that of the product obtained in Example 6.

### Example 9

### Synthesis of 3-(4-hydroxyphenyl)-2-methoxypropionic acid

3-(4-Hydroxyphenyl)-2-methoxyacrylic acid (200 mg, 1.02 mmol), Ru₂Cl₄{(S)-h8-binap}₂NEt₃ (4.4 mg, 0.0051 mmol) and sodium methoxide (55.1 mg, 1.02 mmol) were placed in a 100 ml-autoclave, and the atmosphere was substituted by nitrogen gas. After addition of methanol (2.0 mL), hydrogen gas was supplied to a pressure of 5.0 MPa in the reaction system. The mixture was stirred at 60° C. for 6 hours to give the title compound of 58.0% ee as a crude sodium salt in a conversion rate of >99%.

The crude sodium salt was dissolved in water (10 mL), and the solution was washed twice with toluene (10 mL). 1N Hydrochloric acid (20 mL) was added to the aqueous layer, and the mixture was extracted three times with ethyl acetate (20 mL). The combined organic layers were washed with saturated brine, dried over sodium sulfate, concentrated in vacuo, and dried to give the title compound (117 mg) in 59% yield.
¹H NMR δ (CD₃OD): 7.07 (d, J=8.6 Hz, 2H), 6.71 (d, J=8.6 Hz, 2H), 3.93 (dd, J=4.8, 7.6 Hz, 1H), 3.33 (s, 3H), 2.99 (dd, J=4.8, 14.0 Hz, 1H), 2.85 (dd, J=7.6, 14.0 Hz, 1H).

### Example 10

### Synthesis of Sodium 3-(4-hydroxyphenyl)-2-methoxypropionate

3-(4-Hydroxyphenyl)-2-methoxypropionic acid obtained in Example 9 was dissolved in methanol (2 mL), and to this solution was added 1N sodium hydroxide (0.6 mL). The mixture was stirred at room temperature for 0.5 hours and concentrated in vacuo to give the title compound (138 mg).
¹H NMR δ (CD₃OD) 7.07 (d, J=8.6 Hz, 2H), 6.67 (d, J=8.6 Hz, 2H), 3.69 (dd, J=3.8, 8.6 Hz), 3.24 (s, 3H), 2.94 (dd, J=3.8, 14.0 Hz, 1H), 2.75 (dd, J=8.6, 14.0 Hz, 1H).

### Example 11

### Crystallization of 3-(4-hydroxyphenyl)-2-methoxypropionic acid cyclohexylamine salt

3-(4-Hydroxyphenyl)-2-methoxyacrylic acid (20 g, 0.103 mol) and [RuCl(p-cymene)((S)-dm-segphos)]Cl (0.106 g) were placed in a 1L autoclave, and air in the autoclave was substituted for nitrogen gas. After addition of methanol (200 mL) and cyclohexylamine (12 mL, 0.105 mol), hydrogen gas of 4.0 MPa was introduced to the sealed reaction system, and the mixture was stirred at 80° C. for 16 hours. The reaction mixture was cooled down and methanol was removed by evaporation in vacuo with a rotary evaporator to give a reaction mixture (30.2 g) with conversion rate of >99% and optical purity of >88.6% ee).

To the resultant reaction mixture were added methanol (30 mL) and ethanol (30 mL), and the mixture was heated under ref lux at 95° C., then cooled in an ice-bath. The resultant crystals were collected by filtration to give 3-(4-hydroxyphenyl)-2-methoxypropionic acid cyclohexylamine salt with optical purity of >98% ee.

### Example 12

### Crystallization of sodium 3-(4-hydroxyphenyl)-2-methoxypropionate

3-(4-Hydroxyphenyl)-2-methoxyacrylic acid (20 g, 0.103 mol), sodium methoxide (5.86 g) and [{RuCl((S)-dm-segphos)}₂(μ-Cl)₃]Cl (96.3 mg) were placed in a 1L autoclave, and air in the autoclave was substituted for nitrogen gas. After addition of methanol (200 mL), hydrogen gas of 5.0 MPa was introduced to the sealed reaction system, and the mixture was stirred at 70° C. for 8 hours to give sodium 3-(4-hydroxyphenyl)-2-methoxypropionate with 92.3% ee (conversion rate of >99%). The reaction mixture was cooled down and the resultant product was recrystallized twice from methanol/methyl isobutyl ketone (MIBK) to give sodium 3-(4-hydroxyphenyl)-2-methoxypropionate with optical purity of >99% ee.

### INDUSTRIAL APPLICABILITY

The process of the present invention can provide optically active 3-(4-hydroxyphenyl)propionic acids useful as intermediates for medicines, agrochemicals, etc. Such optically active 3-(4-hydroxyphenyl)propionic acids can be produced through short steps via intermediate cinnamic acids in high yield and in high optical purity.

1. A process for producing an optically active 3-(4-hydroxyphenyl)propionic acid of the formula (6): 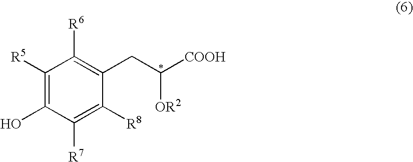
wherein R² is an alkyl group, R⁵ to R⁸ are each independently a hydrogen atom or a substituent; and the symbol * is a chiral carbon atom,
or a salt thereof, which comprises reacting a benzaldehyde of the formula (1): 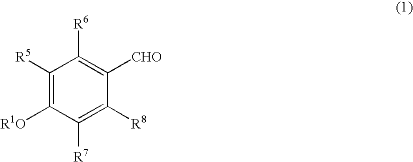
wherein R¹ is a protective group; and R⁵ to R⁸ are each the same as defined above,
with a glycolic acid derivative of the formula (2): 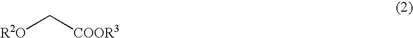
wherein R³ is a hydrocarbon group, and R² is the same as defined above,
hydrolyzing the resulting product to give a cinnamic acid of the formula (4): 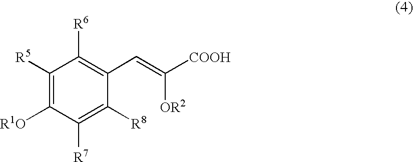
wherein R¹, R², and R⁵ to R⁸ are each the same as defined above,
or a salt thereof, and subjecting the cinnamic acid (4) or a salt thereof to asymmetric hydrogenation to give an optically active phenylpropionic acid of the formula (5): 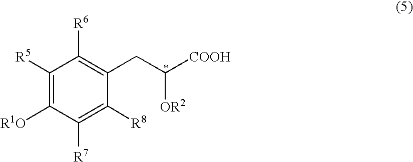
wherein all the symbols are each the same as defined above,
or a salt thereof, followed by deprotection.

2. A process for producing an optically active 3-(4-hydroxyphenyl)propionic acid of the formula (6): 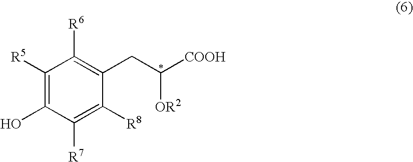
wherein R² is an alkyl group; R⁵ to R⁸ are each independently a hydrogen atom or a substituent;
and the symbol * is a chiral carbon atom,
or a salt thereof, which comprises reacting a benzaldehyde of the formula (1): 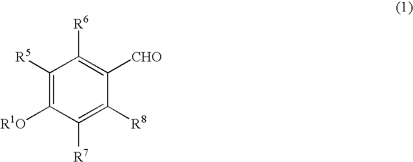
wherein R¹ is a protective group; and R⁵ to R⁸ are each the same as defined above,
with a glycolic acid derivative of the formula (2): 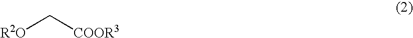
wherein R³ is a hydrocarbon group, and R² is the same as defined above, followed by hydrolysis to give a cinnamic acid of the formula (4): 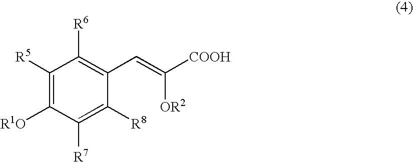
wherein R¹, R², and R⁵ to R⁸ are each the same as defined above,
or a salt thereof, and subjecting the cinnamic acid (4) or a salt thereof to asymmetric hydrogenation.

3. A process for producing an optically active 3-(4-hydroxyphenyl)propionic acid of the formula (6): 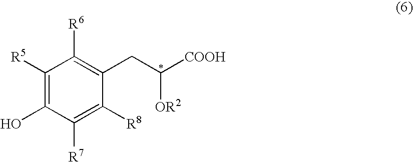
wherein R² is an alkyl group; R⁵ to R⁸ are each independently a hydrogen atom or a substituent;
and the symbol * is a chiral carbon atom,
or a salt thereof, which comprises reacting a 4-hydroxybenzaldehyde of the formula (7): 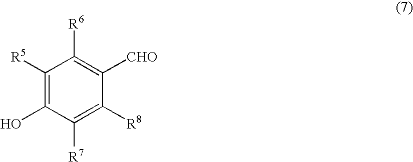
wherein R⁵ to R⁸ are each the same as defined above,
with a glycolic acid derivative of the formula (2): 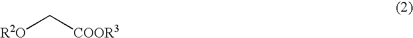
wherein R³ is a hydrocarbon group; and R² is the same as defined above, followed by hydrolysis to give a 4-hydroxycinnamic acid of the formula (9): 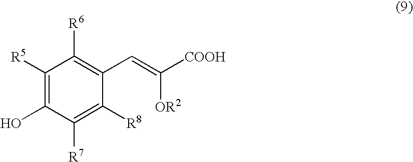
wherein R², and R⁵ to R⁸ are each the same as defined above,
or a salt thereof, and subjecting the 4-hydroxycinnamic acid (9) or a salt thereof to asymmetric hydrogenation.

4. The process according claim 1, wherein the asymmetric hydrogenation is carried out in the presence of a chiral catalyst.

5. The process according claim 1, wherein the chiral catalyst is a transition metal complex.

6. The process according to claim 5, wherein the transition metal complex is a complex of the metal of Groups 8 to 10 in the periodic table.

7. A process for producing an optically active carboxylic acid of the formula (12): 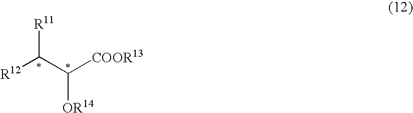
wherein R¹¹ and R¹² are each independently a hydrogen atom or a substituent; R¹³ is a hydrogen atom, an optionally substituted hydrocarbon group or a metal atom; R¹⁴ is a hydrogen atom or a protective group; and the symbol * is an chiral carbon atom, or a salt thereof, which comprises subjecting an (α,β-unsaturated carboxylic acid of the formula (11): 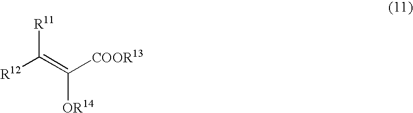
wherein R¹ ¹ to R¹⁴ are each the same as defined above,
or a salt thereof, to asymmetric hydrogenation in the presence of a transition metal complex,
provided that when the transition metal complex is rhodium, the protective group represented by R¹⁴ in the above formula (11) is a group other than acyl.

8. The process according to claim 7, wherein the transition metal complex is a complex of the metal of Groups 8 to 10 in the periodic table.

9. The process according to claim 1, wherein the chiral catalyst is a mixture of a chiral ligand and a transition metal compound.

10. The process according to claim 1, wherein the optically active phenylpropionic acid of the formula (5) or a salt thereof obtained by the method according to claim 1 is crystallized from a solvent.

11. The process according to claim 10, wherein the solvent used for the crystallization is a member selected from the group consisting of hydrocarbons, alcohols, ketones and water, and a mixture thereof.

12. The process according to claim 1, wherein the optically active 3-(4-hydroxyphenyl)propionic acid of the formula (6) or a salt thereof obtained by the method according to claim 1 is crystallized from a solvent.

13. The process according to claim 12, wherein the solvent used for the crystallization is a member selected from the group consisting of aromatic hydrocarbons, aliphatic hydrocarbons, alcohols and water, and a mixture thereof.

14. A process for producing an optically active phenylpropionic acid of the formula (5): 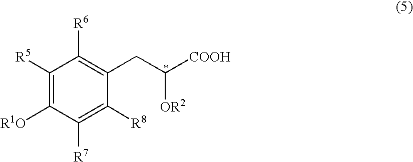
wherein R¹ is a protective group; R² is an alkyl group; R⁵ to R⁸ are each independently a hydrogen atom or a substituent; and the symbol * is an chiral carbon atom,
or a salt thereof
which comprises subjecting a cinnamic acid of the formula (4): 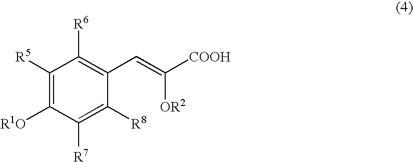
wherein R¹, R², and R⁵ to R⁸ are each the same as defined above,
or a salt thereof,
to asymmetric hydrogenation.

15. A process for producing an optically active 3-(4-hydroxyphenyl)propionic acid of the formula (6): 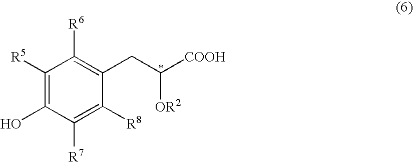
wherein R² is an alkyl group; R⁵ to R⁸ are each independently a hydrogen atom or a substituent;
and the symbol * is a chiral carbon atom,
or a salt thereof, which comprises subjecting a cinnamic acid of the formula (4): 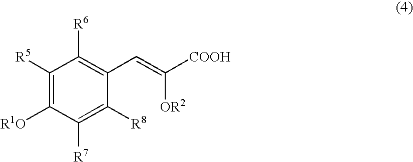
wherein R¹, R², and R⁵ to R⁸ are each the same as defined above,
or a salt thereof, to asymmetric hydrogenation.

16. A process for producing an optically active 3-(4-hydroxyphenyl)propionic acid of the formula (6): 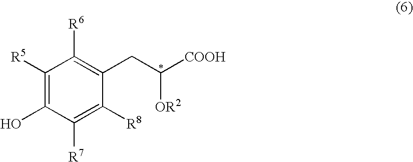
wherein R² is an alkyl group; R⁵ to R⁸ are each independently a hydrogen atom or a substituent;
and the symbol * is a chiral carbon atom,
or a salt thereof,
which comprises subjecting a 4-hydroxycinnamic acid of the formula (9): 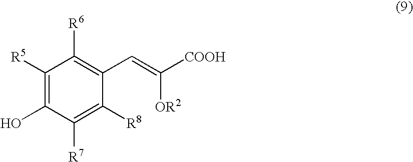
wherein R², and R⁵ to R⁸ are each the same as defined above,
or a salt thereof to asymmetric hydrogenation.

17. A process for producing an optically active 3-(4-hydroxyphenyl)propionic acid of the formula (6): 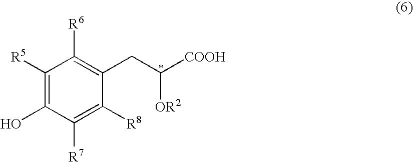
wherein R² is an alkyl group; R⁵ to R⁸ are each independently a hydrogen atom or a substituent;
and the symbol * is a chiral carbon atom,
or a salt thereof, and an optically active phenylpropionic acid of the formula (5): 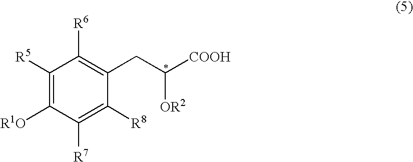
wherein R¹ is a protective group; and R², R⁵ to R⁸ and the symbol * are each the same as defined above,
or a salt thereof, which comprises subjecting a cinnanic acid of the formula (4): 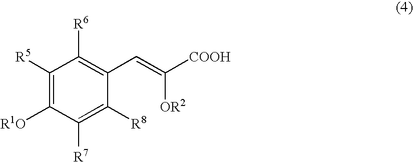
wherein R¹, R², and R⁵ to R⁸ are each the same as defined above,
or a salt thereof, to asymmetric hydrogenation.

18. A process for producing an optically active 3-(4-hydroxyphenyl)propionic acid of the formula (6): 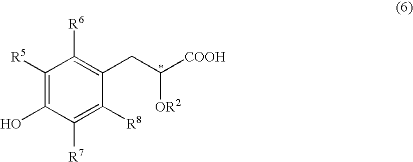
wherein R² is an alkyl group, R⁵ to R⁸ are each independently a hydrogen atom or a substituent;
and the symbol * is a chiral carbon atom,
or a salt thereof, which comprises reacting a benzaldehyde of the formula (1): 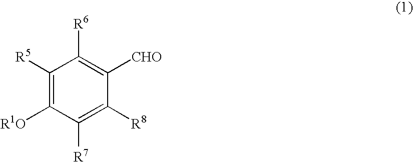
wherein R¹ is a protective group; and R⁵ to R⁸ are each the same as defined above,
with a glycolic acid derivative of the formula (2): 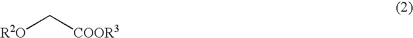
wherein R³ is a hydrocarbon group, and R² is the same as defined above,
hydrolyzing the resulting product to give a cinnamic acid of the formula (4): 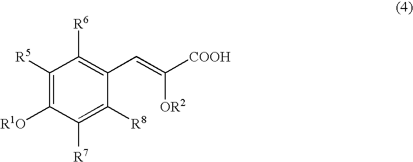
wherein R¹, R², and R⁵ to R⁸ are each the same as defined above,
or a salt thereof, and subjecting the cinnamic acid (4) or a salt thereof to asymmetric hydrogenation to give an optically active phenylpropionic acid of the formula (5): 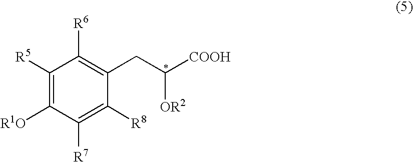
wherein all the symbols are each the same as defined above,
or a salt thereof, and an optically active 3-(4-hydroxyphenyl)propionic acid of the formula (6): 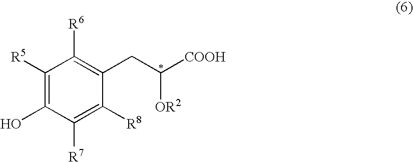
wherein all the symbols are each the same as defined above,
or a salt thereof, followed by deprotection.
